(19) United States
(12) Patent Application Publication
(10) Pub. No.: US 2025/0154188 A1
(43) Pub. Date: May 15, 2025

(54) C-GLYCOSIDES AS ANTI-INFLAMMATORY AGENTS

(71) Applicants: UNIVERSITY OF PITTSBURGH - OF THE COMMONWEALTH SYSTEM OF HIGHER EDUCATION, Pittsburgh, PA (US); THE JOHNS HOPKINS UNIVERSITY, Baltimore, MD (US)

(72) Inventors: Peter Wipf, Pittsburgh, PA (US); Adam Patrick Montoya, San Diego, CA (US); David Hackam, Pittsburgh, PA (US); Chhinder Sodhi, Sewickley, PA (US); Peng Lu, Lutherville Timonium, MD (US)

(73) Assignees: UNIVERSITY OF PITTSBURGH - OF THE COMMONWEALTH SYSTEM OF HIGHER EDUCATION, Pittsburgh, PA (US); THE JOHNS HOPKINS UNIVERSITY, Baltimore, MD (US)

(21) Appl. No.: 19/011,924

(22) Filed: Jan. 7, 2025

Related U.S. Application Data
(63) Continuation of application No. PCT/US2023/027178, filed on Jul. 7, 2023.
(60) Provisional application No. 63/443,835, filed on Feb. 7, 2023.
(60) Provisional application No. 63/426,704, filed on Nov. 18, 2022.
(60) Provisional application No. 63/359,049, filed on Jul. 7, 2022.

(51) Int. Cl. C07H 3/02; A61K 31/7004; A61K 31/7008; C07H 5/06

(52) U.S. Cl. CPC C07H 3/02; A61K 31/7004; A61K 31/7008; C07H 5/06

(57) ABSTRACT

Novel compounds and methods of using the compounds are provided herein. The compounds include novel carbon analogs of pyranose derivatives discovered to have Toll-like receptor 4 (Tlr4) inhibitory activity. The methods provide for treating infectious, inflammatory and post-traumatic disorders.

### CROSS-REFERENCE TO RELATED APPLICATIONS

This application is a continuation of International Application No. PCT/US2023/027178 filed on Jul. 7, 2023, which claims the benefit of priority to U.S. Provisional Patent Application Ser. No. 63/359,049 filed on Jul. 7, 2022, U.S. Provisional Patent Application Ser. No. 63/426,704 filed on Nov. 18, 2022, and U.S. Provisional Patent Application Ser. No. 63/443,835 filed on Feb. 7, 2023, the contents of which are incorporated herein by reference in their entireties.

### GRANT INFORMATION

This disclosure was made with government support under grant numbers R01DK117186, R01DK083752 and R01GM078238 awarded by the National Institutes of Health. The government has certain rights in the invention.

### 1. INTRODUCTION

The presently disclosed subject matter relates to novel carbon analogs of pyranose derivatives discovered to have Tlr4 inhibitory activity. The disclosed subject matter further provides methods for using the pyranose derivatives for treating infectious, inflammatory and post-traumatic disorders.

### 2. BACKGROUND OF THE INVENTION

The innate immune receptor Toll-like receptor 4 (“Tlr4”) has been recognized to be the receptor on hematopoietic and non-hematopoietic cells for endotoxin (lipopolysaccharide, “LPS”) as well as a variety of endogenous molecules that are released within the body during inflammatory or infectious disorders. Because Tlr4 is the most upstream receptor in the pro-inflammatory LPS signaling cascade, use inhibitors that antagonize Tl4 signaling could avoid the pitfalls associated with other cytokine inhibitors that act further downstream in the pathway, and, accordingly, play a less significant role.

### 3. SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter includes carbon analogs of pyranose derivatives and methods for using said derivatives for treating infectious, inflammatory and post-traumatic disorders and have Tlr4 signaling inhibitory activity. Certain compounds that can be used according to the present disclosure are set forth in TABLE 1, below. In addition to methods of treatment, the presently disclosed subject matter further provides for pharmaceutical compositions comprising said compounds, together with a suitable pharmaceutical carrier. Because Tlr4 is the most upstream receptor in the pro-inflammatory LPS signaling cascade, treatments of the disclosed subject matter, which inhibit or antagonize Tlr4 action, can avoid the pitfalls associated with other cytokine inhibitors that act further down the pathway and accordingly play a less specific (and perhaps non-critical) role.

In certain embodiments, the disclosed subject matter provides for a compound of Formula I or stereoisomers thereof:

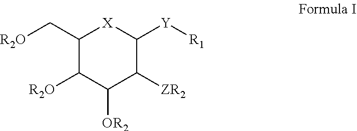

wherein X is O, Z is O or NH, Y is CH₂ or CH(R₁), R₁ is alkyl, branched alkyl, cycloalkyl, aryl, or substituted aryl, and R₂ is H or COR₃, where R₃ is selected from R₁, NHR₁, or N(R₁) 2, or
wherein X is CH₂, Z is O or NH, Y is O, R₁ is alkyl, branched alkyl, cycloalkyl, aryl, or substituted aryl, and R₂ is H or COR₃, where R₃ is selected from R₁, NHR₁, or N(R₁) 2.

The disclosed subject matter also provides for a pharmaceutical compositions comprising a compound of Formula I. In certain embodiments, the pharmaceutical composition is selected from the group consisting of a coated particle, a micelle, a liposome, a tablet, a capsule, a sachet, a suppository, a liquid pharmaceutical composition, and combinations thereof. In certain embodiments, the pharmaceutical composition further comprises an antibiotic agent, a steroid, or a non-steroidal anti-inflammatory agent, or combinations thereof.

In certain embodiments, the compound of Formula I is selected from the group consisting of the following structures:

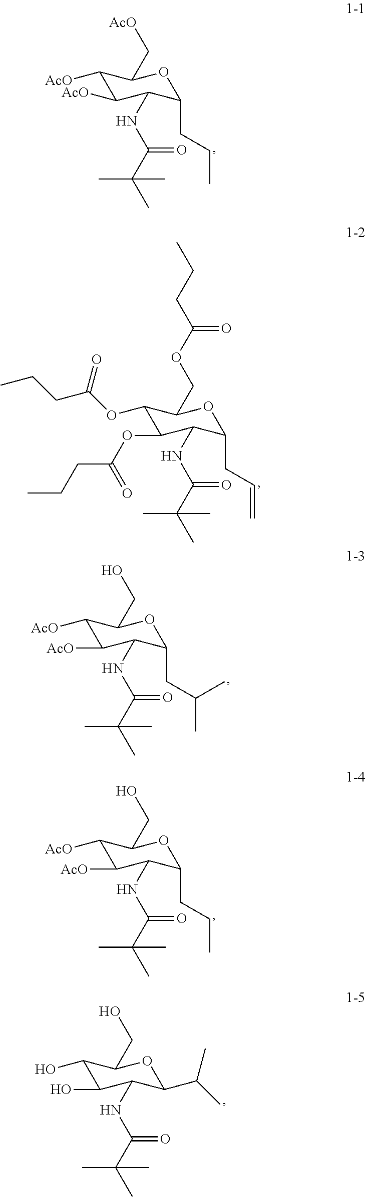

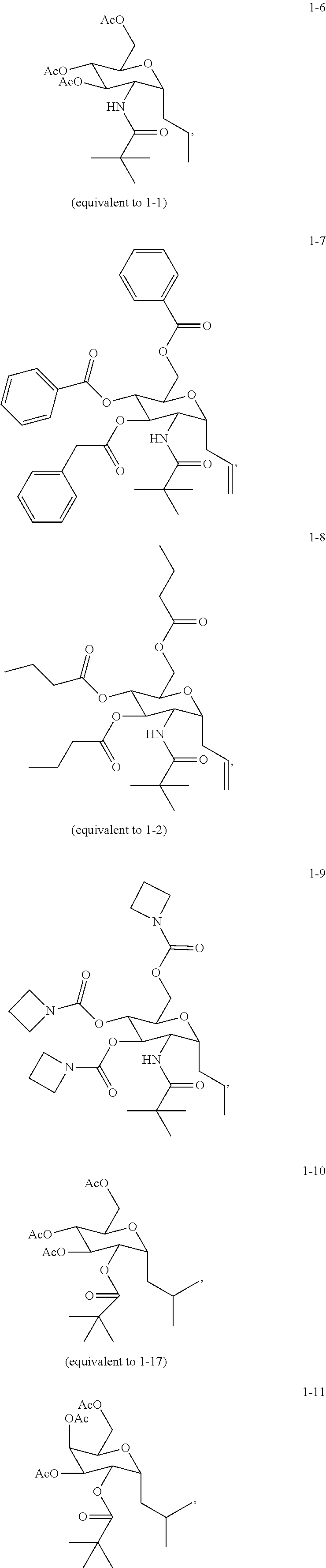

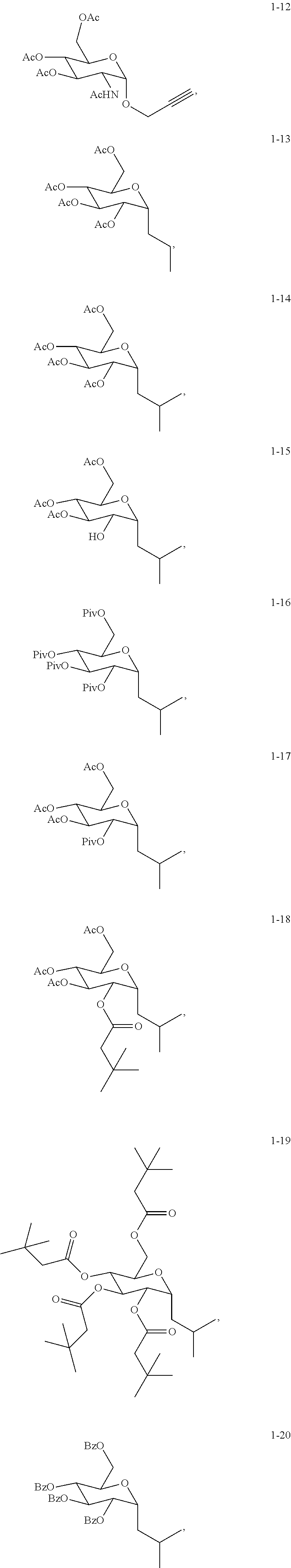

and combinations thereof.

The presently disclosed subject matter also provides for a compound of Formula II or stereoisomers thereof:

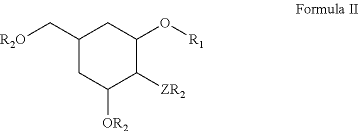

wherein Z is O or NH, R₁ is alkyl, branched alkyl, cycloalkyl, aryl, or substituted aryl, and R₂ is H or COR₃, where R₃ is selected from R₁, NHR₁, or N(R₁)₂.

The disclosed subject matter further provides for pharmaceutical compositions comprising a compound of Formula II. In certain of these embodiments, the pharmaceutical composition is selected from the group consisting of a coated particle, a micelle, a liposome, a tablet, a capsule, a sachet, a suppository, a liquid pharmaceutical composition, and combinations thereof. In certain of these embodiments, the pharmaceutical composition further comprises an antibiotic agent, a steroid, or a non-steroidal anti-inflammatory agent, or combinations thereof.

According to certain embodiments of the presently disclosed subject matter, the compound of Formula II is selected from the group consisting of the following structures:

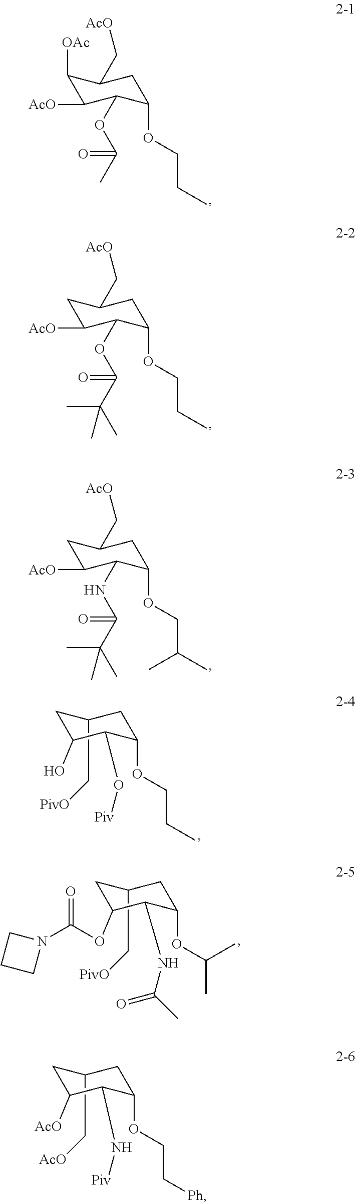

and combinations thereof.

In other embodiments, the disclosed subject matter provides for methods of treating an infectious or inflammatory disorder comprising administering, to a subject in need of such treatment, an effective amount of a Toll-like receptor 4 inhibitor compound of Formula I or stereoisomers thereof:

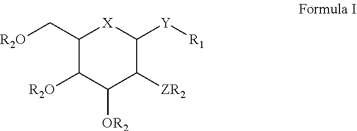

wherein X is O, Z is O or NH, Y is CH₂ or CH(R₁), R₁ is alkyl, branched alkyl, cycloalkyl, aryl, or substituted aryl, and R₂ is H or COR₃, where R₃ is selected from R₁ or NHR₁, N(R₁)₂, or
wherein X is CH₂, Z is O or NH, and Y is O, R₁ is alkyl, branched alkyl, cycloalkyl, aryl, or substituted aryl, and R₂ is H or COR₃, where R₃ is selected from R₁, NHR₁, or N(R₁)₂.

In another embodiment, the disclosed subject matter provides for methods of treating an infectious or inflammatory disorder comprising administering, to a subject in need of such treatment, an effective amount of a Toll-like receptor 4 inhibitor compound of Formula II or stereoisomers thereof:

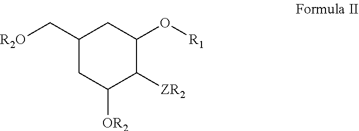

wherein Z is O or NH, with R₁ is alkyl, branched alkyl, cycloalkyl, aryl, or substituted aryl, and R₂ is H or COR₃, where R₃ is selected from R₁, NHR₁, or N(R₁)₂.

In certain embodiments of the methods, the subject is suffering from one or more of osteoarthritis, pancreatitis, a metabolic syndrome, trauma induced systemic inflammation, acute respiratory distress syndrome, and COVID-19-induced systemic inflammation. In specific embodiments, the subject is a suffering from necrotizing enterocolitis.

According to the disclosed methods, the Toll-like receptor 4 inhibitor is selected from the group consisting of:

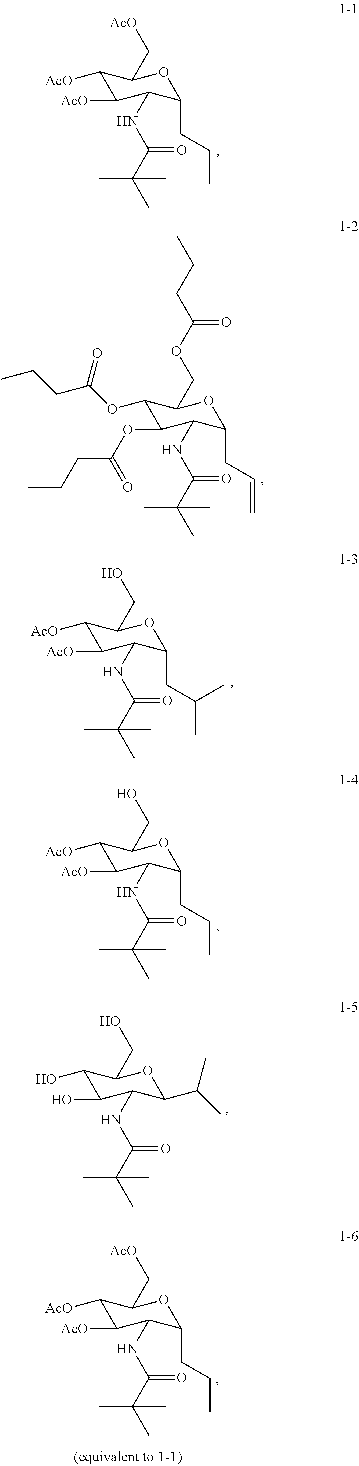

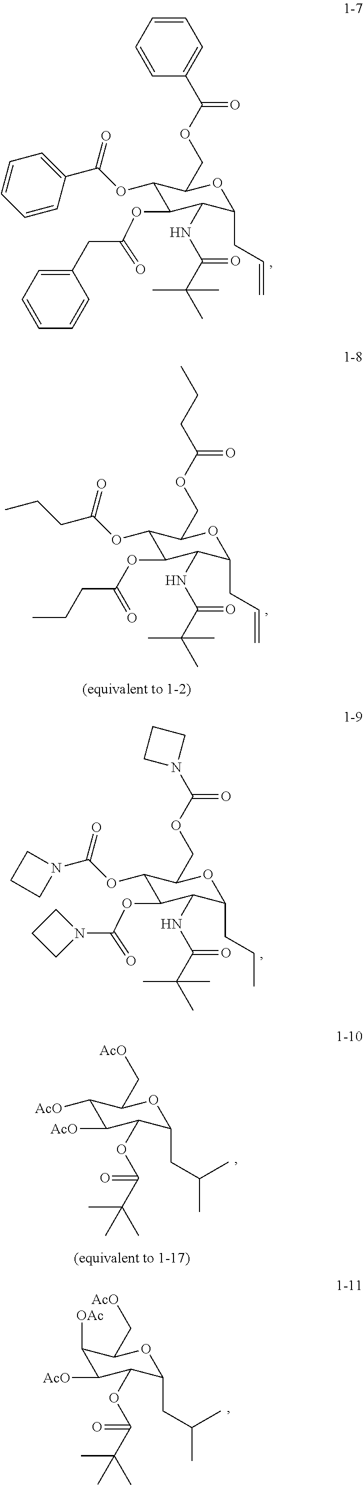

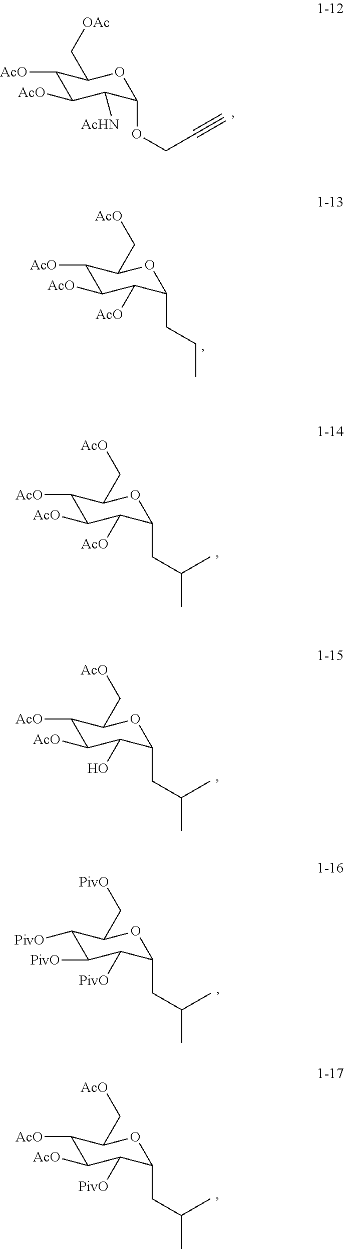

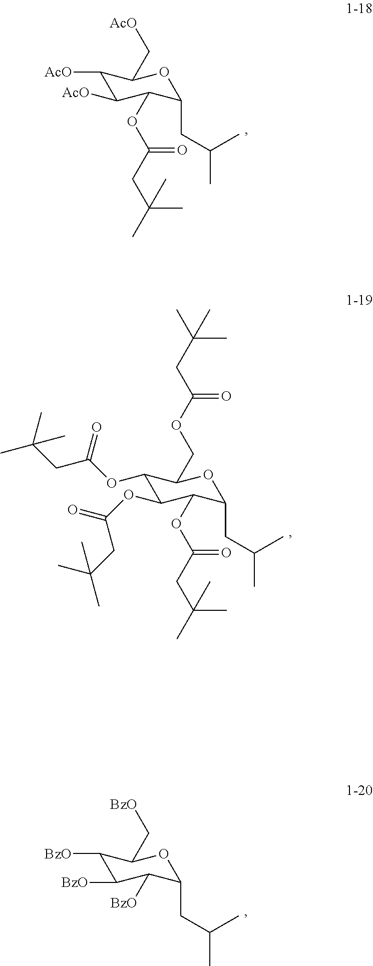

and combinations thereof.

In alternative embodiments, the Toll-like receptor 4 inhibitor is selected from the group consisting of:

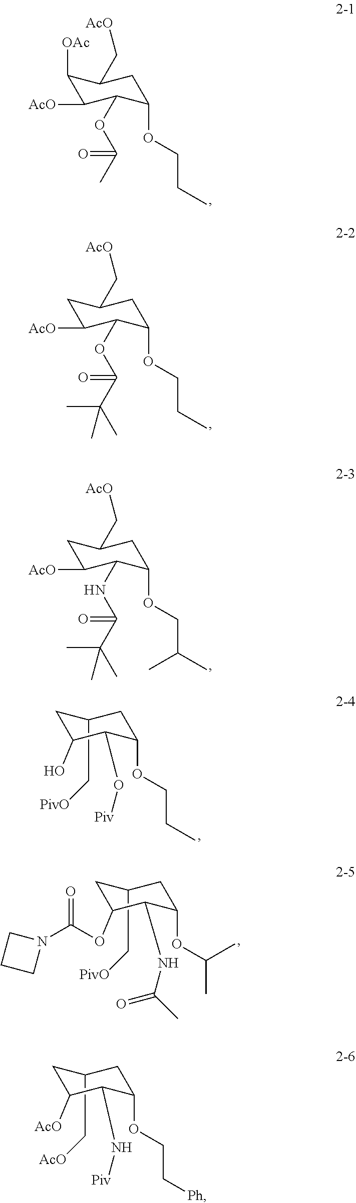

and combinations thereof.

The disclosed subject matter also provides for methods of treating a traumatic injury in a subject comprising administering, to a subject in need of such treatment, an effective amount of a Toll-like receptor 4 inhibitor compound that reduces Toll-like receptor 4-induced post-traumatic injury. In certain embodiments, the traumatic injury is to an organ selected from the group consisting of the heart, the liver, the lung, the kidney, the intestine, the brain, the eye and the pancreas.

In certain embodiments, the compound is administered to treat organ rejection after transplantation.

In certain embodiments, the methods comprise administering a compound of Formula I or Formula II to prevent necrotizing enterocolitis in a premature infant.

### 4. BRIEF DESCRIPTION OF THE DRAWINGS

The following figures are included to illustrate certain aspects of the present disclosure and should not be viewed as exclusive embodiments. The subject matter disclosed is capable of considerable modifications, alterations, combinations, and equivalents in form and function, without departing from the scope of this disclosure.

FIG. 1 provides the relative mRNA expression levels of Toll-like receptor 4 (Tlr4) in mouse enteroids cultures treated with saline (Ctrl), lipopolysaccharide (LPS) or LPS with test compounds (C277, C278, C279, C280, C281, C282, C283, C284).

FIG. 2 provides the relative mRNA expression levels of tumor necrosis factor (Tnf) in mouse enteroids cultures treated with saline (Ctrl), lipopolysaccharide (LPS) or LPS with test compounds (C277, C278, C279, C280, C281, C282, C283, C284).

FIG. 3 provides the relative mRNA expression levels of Toll-like receptor 4 (Tlr4) in the ileum of healthy control neonatal mice (Ctrl), mice with NEC or mice with NEC treated with test compounds (C277, C278, C280, C281, C282, C283, C284). Statistical significance is indicated by *p<0.05 p values.

FIG. 4 provides the relative mRNA expression levels of interleukin 6 (Il6) in the ileum of healthy control neonatal mice (Ctrl), mice with NEC or mice with NEC treated with test compounds (C277, C278, C280, C281, C282, C283, C284). Statistical significance is indicated by *p<0.05 p values.

FIG. 5 provides the relative mRNA expression levels of interleukin 1 beta (Il1b) in the ileum of healthy control neonatal mice (Ctrl), mice with NEC or mice with NEC treated with test compounds (C277, C278, C280, C281, C282, C283, C284). Statistical significance is indicated by *p<0.05 p values.

FIG. 6 provides the relative provides the relative mRNA expression levels of tumor necrosis factor (Tnf) in the ileum of healthy control neonatal mice (Ctrl), mice with NEC or mice with NEC treated with test compounds (C277, C278, C280, 5 C281, C282, C283, C284). Statistical significance is indicated by *p<0.05, **p<0.01, ****p<0.0001 p values.

FIG. 7 provides the relative provides the relative mRNA expression levels of lipocalin 2 (Lcn2) in the ileum of healthy control neonatal mice (Ctrl), mice with NEC or mice with NEC treated with test compounds (C277, C278, C280, C281, C282, C283, C284). Statistical significance is indicated by *p<0.05, ***p<0.001 p values.

FIG. 8 provides the relative mRNA expression levels of Toll-like receptor 4 (Tlr4) in the ileum of neonatal mice treated with saline (Sal) or lipopolysaccharide (LPS), or LPS with test compounds (C277, C278, C279, C280, C281, C282, C283, C284). Statistical significance is indicated by *p<0.05, **p<0.01 p values.

FIG. 9 provides the relative mRNA expression levels of interleukin 6 (Il6) in the ileum of neonatal mice treated with saline (Sal) or lipopolysaccharide (LPS), or LPS with test compounds (C277, C278, C279, C280, C281, C282, C283, C284). Statistical significance is indicated by *p<0.05, **p<0.01, ****p<0.0001 p values.

FIG. 10 provides the relative mRNA expression levels of interleukin 1 beta (Il1b) in the ileum of neonatal mice treated with saline (Sal) or lipopolysaccharide (LPS), or LPS with test compounds (C277, C278, C279, C280, C281, C282, C283, C284). Statistical significance is indicated by **p<0.01, ***p<0.001, ****p<0.0001 p values.

FIG. 11 provides the relative mRNA expression levels of tumor necrosis factor (Tnf) in the ileum of neonatal mice treated with saline (Sal) or lipopolysaccharide (LPS), or LPS with test compounds (C277, C278, C279, C280, C281, C282, C283, C284). Statistical significance is indicated by ***p<0.001, ****p<0.0001 p values.

FIG. 12 provides the relative mRNA expression levels of lipocalin 2 (Lcn2) in the ileum of neonatal mice treated with saline (Sal) or lipopolysaccharide (LPS), or LPS with test compounds (C277, C278, C279, C280, C281, C282, C283, C284). Statistical significance is indicated by *p<0.05, ***p<0.001, ****p<0.0001 p values.

FIG. 13 provides hematoxylin and eosin (H&E) representative images of non-NEC control, NEC, and NEC+C281 ileal tissue sections.

### 5. DETAILED DESCRIPTION OF THE INVENTION

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. Moreover, the term “alkyl” (or “lower alkyl”) includes both “unsubstituted alkyls” and “substituted alkyls,” the latter of which refers to alkyl moieties having substituents replacing hydrogen on one or more carbons of the hydrocarbon backbone. Such substituents may include, for example, a halogen, a hydroxyl, a carbonyl (such as a carboxyl, an alkoxycarbonyl, a formyl or an acyl), a thiocarbonyl (such as a thioester, a thioacetate, or a thioformate), an alkoxyl, a phosphoryl, a phosphonate, a phosphinate, an amino, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain may themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls may be further substituted with alkyls, alkenyls, alkoxys, alkylthios, aminoalkyls, carbonyl-substituted alkyls, —CF₃, —CN and the like.

The terms “amine” and “amino” are art-recognized and include both unsubstituted and substituted amines. A primary amine carries two hydrogens, a secondary amine, one hydrogen and another substituent and a tertiary amine, the two hydrogens are substituted. The substituents for one or both of the hydrogens can be, for example, and alkyl, an alkenyl, and aryl, a cycloalkyl, a cycloalkenyl, a heterocycle, a polycycle and so on. If both hydrogens are substituted with carbonyls, the carbonyl framed nitrogen forms an imide.

The term “alkylamine” includes an amine group, as defined above, having a substituted or unsubstituted alkyl attached thereto.

The term “aryl” is art-recognized, and includes 5-, 6-, and 7-membered single ring aromatic groups that may include from zero to four heteroatoms, for example, benzene, pyrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls, and/or heterocyclyls, or rings joined by non-cyclic moieties.

The terms “heterocyclyl” and “heterocyclic group” are art-recognized, and include 3- to about 10-membered ring structures, such as 3- to about 7-membered rings, whose ring structures include one to four heteroatoms. Heterocycles may also be polycycles. Heterocyclyl groups include, for example, thiophene, thianthrene, furan, pyran, isobenzofuran, chromene, xanthene, phenoxanthin, pyrrole imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphtyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones and the like. The heterocyclic ring may be substituted at one or more positions with such substituents as described above, as for example, halogen, alkyl aralkyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CD₃, —CN or the like.

The terms “polycyclyl” and polycyclic group” are art-recognized and include structures with two or more rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms, e.g., three or more atoms are common to both rings, are termed “bridged” rings. Each of the rings of the polycycle may be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CD₃, —CN or the like.

The term “carbocycle” is art recognized and includes an aromatic or non-aromatic ring in which each atom of the ring is carbon. The following art-recognized terms have the following meanings: “nitro” means —NO₂, the term “halogen” designates —F, —Cl, —Br, or —I; the term “sulfhydryl” means —SH; the term “hydroxyl” or “hydroxy” means —OH; and the term sulfonyl” means —SO₂—.

The terms “amine” and “amino” are art-recognized and include both unsubstituted and substituted amines. A primary amine carries two hydrogens, a secondary amine, one hydrogen and another substituent and a tertiary amine, the two hydrogens are substituted. The substituents for one or both of the hydrogens can be, for example, and alkyl, an alkenyl, and aryl, a cycloalkyl, a cycloalkenyl, a heterocycle, a polycycle and so on. If both hydrogens are substituted with carbonyls, the carbonyl framed nitrogen forms an imide.

The term “alkylamine” includes an amine group, as defined above, having a substituted or unsubstituted alkyl attached thereto.

The term “amido” is art-recognized as an amino-substituted carbonyl.

The term “alkylthio” is art-recognized and includes and alkyl group, as defined above, having a sulfur radical attached thereto. In certain embodiments, the “alkylthio” moiety is represented by one of —S-alkyl, —S-alkenyl, —S-alkynyl and so on.

The term “carbonyl” is art-recognized and includes a C═O structure. Carbonyls are involved in esters; carboxyl groups; formates; thiocarbonyls; thioesters; thiocarboxylic acids; thioformates; ketones; and aldehydes.

The terms “alkoxyl” and “alkoxy” are art-recognized and include an alkyl group, as defined above, having an oxygen radical attached thereto. Representative alkoxyl groups include methoxy, ethoxy, propyloxy, tert-butoxy and the like.

An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl and so on.

The term “sulfonate” is art-recognized and includes a moiety wherein a sulfur atom carries two double bonded oxygens and a single bonded oxygen.

The term “sulfate” is art-recognized and includes a moiety that resembles a sulfonate but includes two single bonded oxygens.

The terms “sulfonamide,” “sulfamoyl,” “sulfonyl,” and “sulfoxido” are art-recognized and each can include a variety of R group substituents as described herein.

The terms phosphoramidite” and “phophonamidite” are art-recognized.

The term “selenoalkyl” is art-recognized and includes an alkyl group having a substituted seleno group attached thereto. Exemplary “selenoethers” which may be substituted on the alkyl are selected from one of —Se-alkyl, —Se-alkenyl, —Se-alkynyl and so on.

Substitutions may be made to alkenyl and alkynyl groups to produce, for example, aminoalkenyls, aminoalkynyls, amidoalkenyls, iminoalkenyls, iminoalkynyls, thioalkenyls, thioalkynyls, carbonyl-substituted alkenyls or alkynyls.

It will be understood that “substitution” or “substituted with” includes the implicit proviso that such substitution is in accordance with the permitted valency of the substituted atom and the substituent, and that the substitution results in a stable compound, e.g., which does not spontaneously undergo transformation, such as by rearrangement, cyclization, elimination, or other reaction.

The term “substituted” is also contemplated to include all permissible substituents of organic compounds such as the imide reagent of interest. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents may be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

As used herein, the term “enantiomers” refers to a pair of stereoisomers that are non-superimposable mirror images of each other. A 1:1 mixture of a pair of enantiomers is a “racemic” mixture. The term is used to designate a racemic mixture where appropriate.

As used herein, the term “diastereoisomers” refers to stereoisomers that have at least two asymmetric atoms, but which are not mirror-images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line. The compounds of the presently disclosed subject matter contain one or more asymmetric centers and can thus give rise to enantiomers, diastereomers, and other stereoisomeric forms that can be defined, in terms of absolute stereochemistry, as (R)- or (S)-. The presently disclosed subject matter is meant to include all such possible isomers, including racemic mixtures, optically pure forms, and intermediate mixtures. Optically active (R)- and (S)-isomers can be prepared using chiral synthons or chiral reagents, or resolved using conventional techniques. If the compound contains a double bond, the substituent can be E or Z configuration. If the compound contains a disubstituted cycloalkyl, the cycloalkyl substituent can have a cis- or trans-configuration. All tautomeric forms are also intended to be included.

As used herein, the term “isomers” refers to different compounds that have the same molecular formula but differ in arrangement and configuration of the atoms. Also as used herein, the term “stereoisomer” refers to any of the various stereo isomeric configurations which may exist for a given compound of the presently disclosed subject matter and includes geometric isomers. It is understood that a substituent may be attached at a chiral center of a carbon atom. Therefore, the presently disclosed subject matter includes enantiomers, diastereomers, or racemates of the compound. Also, as used herein, the terms “constitutional isomers” refers to different compounds which have the same numbers of, and types of, atoms but the atoms are connected differently.

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

For purposes of clarity and not by way of limitation, the detailed description of the disclosed subject matter is divided into the following subsections:
5.1 Tlr4 inhibitory compounds;
5.2 pharmaceutical compositions;
5.3 disorders; and
5.4 methods of treatment.

### 5.1 TLR4 INHIBITORY COMPOUNDS

The present disclosure provides carbon analogs of pyranose derivatives. In certain embodiments, said compounds can be used to inhibit Tlr4.

In certain embodiments, the present disclosure provides a compound of Formula I:

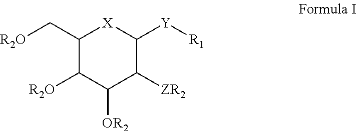

wherein X is O, Z is O or NH, Y is CH₂ or CH(R₁), R₁ is alkyl, branched alkyl, cycloalkyl, aryl, or substituted aryl, and R₂ is H or COR₃, where R₃ is selected from R₁, NHR₁, or N(R₁)₂, or
wherein X is CH₂, Z is O or NH, and Y is O, R₁ is alkyl, branched alkyl, cycloalkyl, aryl, or substituted aryl, and R₂ is H or COR₃, where R₃ is selected from R₁, NHR₁, or N(R₁)₂.

In certain embodiments, the present disclosure provides a compound of Formula II:

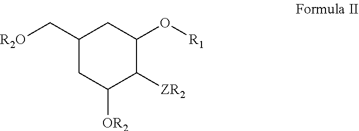

wherein Z is O or NH, R₁ is alkyl, branched alkyl, cycloalkyl, aryl, or substituted aryl, and R₂ is H or COR₃, where R₃ is selected from R₁, NHR₁, or N(R₁)₂.

In certain specific embodiments, the compounds of the present disclosure are provided in Table 1.

TABLE 1

| Compound No. | Structure |
|---|---|
| 1-1 | 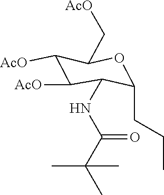 [SMILES: CCCC1OC(COC(C)=O)C(OC(C)=O)C(OC(C)=O)C1NC(=O)C(C)(C)C] |
| 1-2 | 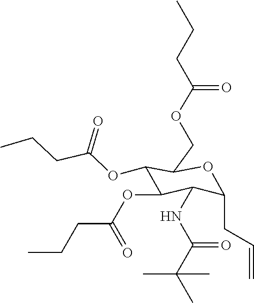 [SMILES: C=CCC1OC(COC(=O)CCC)C(OC(=O)CCC)C(OC(=O)CCC)C1NC(=O)C(C)(C)C] |
| 1-3 | 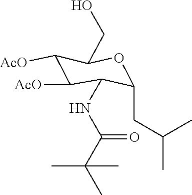 [SMILES: CC(=O)OC1C(CO)OC(CC(C)C)C(NC(=O)C(C)(C)C)C1OC(C)=O] |
| 1-4 | 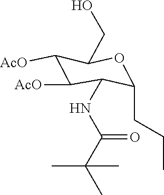 [SMILES: CCCC1OC(CO)C(OC(C)=O)C(OC(C)=O)C1NC(=O)C(C)(C)C] |
| 1-5 | 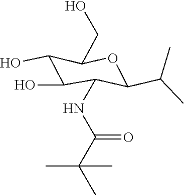 [SMILES: CC(C)C1OC(CO)C(O)C(O)C1NC(=O)C(C)(C)C] |
| 1-6 | 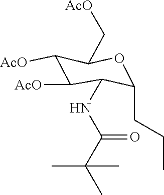 [SMILES: CCCC1OC(COC(C)=O)C(OC(C)=O)C(OC(C)=O)C1NC(=O)C(C)(C)C] |
| | (equivalent to 1-1) |
| 1-7 | 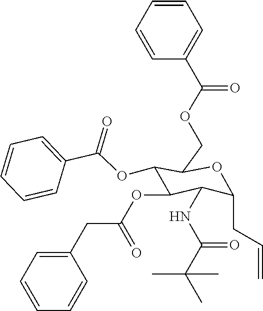 [SMILES: C=CCC1OC(COC(=O)c2ccccc2)C(OC(=O)c2ccccc2)C(OC(=O)Cc2ccccc2)C1NC(=O)C(C)(C)C] |
| 1-8 | 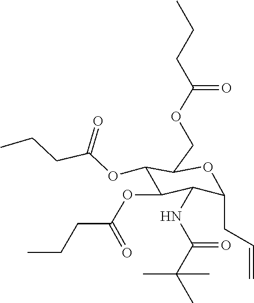 [SMILES: C=CCC1OC(COC(=O)CCC)C(OC(=O)CCC)C(OC(=O)CCC)C1NC(=O)C(C)(C)C] |
| | (equivalent to 1-2) |
| 1-9 | 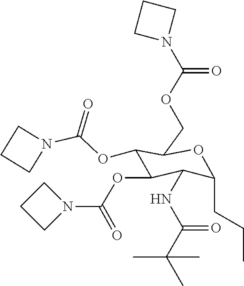 [SMILES: CCCC1OC(COC(=O)N2CCC2)C(OC(=O)N2CCC2)C(OC(=O)N2CCC2)C1NC(=O)C(C)(C)C] |
| 1-10 | 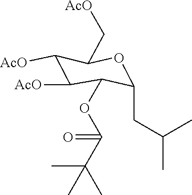 [SMILES: CC(=O)OCC1OC(CC(C)C)C(OC(=O)C(C)(C)C)C(OC(C)=O)C1OC(C)=O] |
| | (equivalent to 1-17) |
| 1-11 | 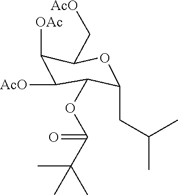 [SMILES: CC(=O)OCC1OC(CC(C)C)C(OC(=O)C(C)(C)C)C(OC(C)=O)C1OC(C)=O] |
| 1-12 | 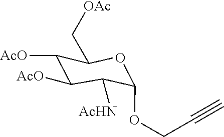 [SMILES: C#CCOC1OC(COC(C)=O)C(OC(C)=O)C(OC(C)=O)C1NC(C)=O] |
| 1-13 [C277] | 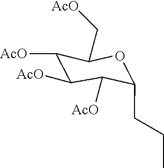 [SMILES: CCCC1OC(COC(C)=O)C(OC(C)=O)C(OC(C)=O)C1OC(C)=O] |
| 1-14 [C278] | 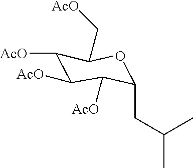 [SMILES: CC(=O)OCC1OC(CC(C)C)C(OC(C)=O)C(OC(C)=O)C1OC(C)=O] |
| 1-15 [C279] | 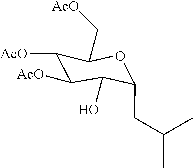 [SMILES: CC(=O)OCC1OC(CC(C)C)C(O)C(OC(C)=O)C1OC(C)=O] |
| 1-16 [C280] | 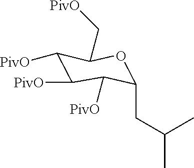 |
| 1-17 [C282] | 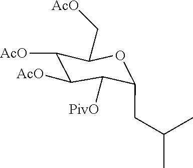 |
| 1-18 [C283] | 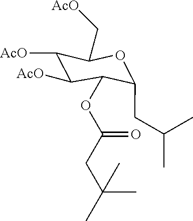 [SMILES: CC(=O)OCC1OC(CC(C)C)C(OC(=O)CC(C)(C)C)C(OC(C)=O)C1OC(C)=O] |
| 1-19 [C281] | 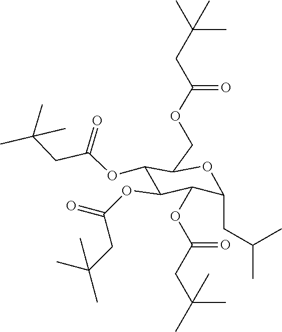 [SMILES: CC(C)CC1OC(COC(=O)CC(C)(C)C)C(OC(=O)CC(C)(C)C)C(OC(=O)CC(C)(C)C)C1OC(=O)CC(C)(C)C] |
| 1-20 [C284] | 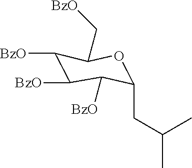 |
| 2-1 | 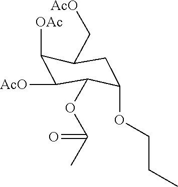 [SMILES: CCCOC1CC(COC(C)=O)C(OC(C)=O)C(OC(C)=O)C1OC(C)=O] |
| 2-2 | 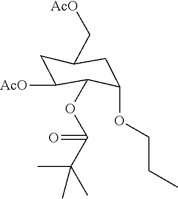 [SMILES: CCCOC1CC(COC(C)=O)CC(OC(C)=O)C1OC(=O)C(C)(C)C] |
| 2-3 | 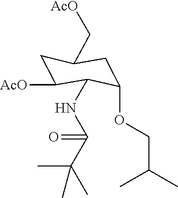 [SMILES: CC(=O)OCC1CC(OCC(C)C)C(NC(=O)C(C)(C)C)C(OC(C)=O)C1] |
| 2-4 | 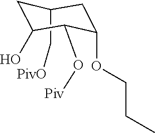 |
| 2-5 | 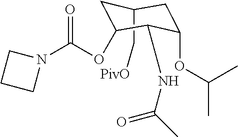 |
| 2-6 | 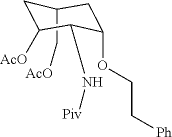 |

In certain embodiments, a compound is considered to inhibit Tlr4 if it inhibits one or more sign or symptom of inflammation, such as, for example, activation of nuclear factor kappa light chain enhancer of activated B cells (“NFkB”), increased expression/levels of interleukins, including but not limited to interleukin 1 (“Il1”) and interleukin 6 (“Il6”), increased expression/levels of tumor necrosis factor (“Tnf”), increased expression/levels of lipocalin-2 (“Lcn2”), elevated erythrocyte sedimentation rate, elevated C reactive protein, fever, tachypnea, lethargy, swelling, redness, and/or pain. In certain embodiments, the ability for a compound and/or a particular concentration of a compound to inhibit Tlr4 can be determined using an assay for Tlr4 activity, which can assess one of the above-listed signs or symptoms. For example, in certain embodiments, Tlr4 inhibition can be assayed using a method that measures the effect of a compound on NFkB activity, for example, but not limited to, the NFkB luciferase reporter mouse model, stimulated with a Tlr4 ligand such as LPS, described in the example below or HEK-Blue-4 cells (InvivoGen). Certain other non-limiting examples of systems for testing compounds to determine Tlr4 inhibitory activity include CWT mice can be treated with LPS and a test compound and monitored for signs and symptoms of inflammation, and/or C3H/WT cells (InvivoGen) can be treated with LPS and a test compound and tested for NFkB activation, Il6 production, or other markers of the inflammatory process.

### 5.2 PHARMACEUTICAL COMPOSITIONS

In certain embodiments, the present disclosure provides for pharmaceutical compositions comprising a compound of Formula I or Formula II, as described above, in a suitable pharmaceutical carrier. The amount of the compound present in the composition can be calculated to provide, when administered to a subject in need of such treatment, an effective amount of the compound of Formula I or Formula II.

In certain embodiments, the present disclosure provides for pharmaceutical compositions including therapeutically effective amounts of any of the compound of Formula I or Formula II, for example but not limited to together with a pharmaceutical carrier such as water or other physiologic solvent. A therapeutically effective amount inhibits Tlr4.

In non-limiting embodiments, the compound of Formula I or Formula II can be included in a coated particle, micelle, liposome, or a similar structure.

In certain embodiments, the pharmaceutical composition can be a liquid, including a compound of Formula I or Formula II in a liquid pharmaceutical carrier including, for example, water (an aqueous carrier) or saline. In certain embodiments, said liquid composition can optionally further contain one or more of a buffer or a preservative.

In certain other embodiments, the pharmaceutical composition of the present disclosure can be a solid, for example in the form of a tablet, capsule, sachet or suppository, including a dose of a compound of Formula I or Formula II that provides an effective amount of the compound of Formula I or Formula II to a subject in need of such treatment when administered according to a dosing regimen. In certain embodiments, said solid pharmaceutical composition can further include one or more excipients, for example, but not limited to, lactose, sucrose, mannitol, erythritol, carboxymethylcellulose, microcrystalline cellulose, hydroxypropylmethylcellulose, starch, polyvinylpyrrolidone, etc.

In certain embodiments, a pharmaceutical composition can include an additional agent that has antimicrobial and/or anti-inflammatory activity. In certain embodiments, such compound includes, but is not limited to, an antibiotic agent, a steroid, or a non-steroidal anti-inflammatory agent. In certain other embodiments, the pharmaceutical composition can include an analgesic agent. In certain further embodiments, the pharmaceutical composition can include an agent that improves cardiac function and/or reduces cardiac stress, for example, but not limited to, an angiotensin converting enzyme inhibitor, a beta blocker, nitroglycerin or a related nitrate compound, digoxin or a related compound, or a calcium channel blocker.

Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66:1-19 (1977).

### 5.3 DISORDERS

In certain embodiments, the compounds, compositions and methods provided herein can be used to treat any disease/disorder (“disorder”) involving Tlr44 activation, including, but not limited to, infectious diseases and inflammatory disorders such as sepsis, necrotizing enterocolitis (“NEC”), autoimmune diseases, Crohn's disease, celiac disease, ulcerative colitis, rheumatoid arthritis, cardiovascular disease including myocardial infarction, epilepsy, gram negative bacterial infections, aspergillosis, periodontal disease, Alzheimer's disease, cigarette smoke mediated lung inflammation, viral hepatitis (including hepatitis C virus hepatitis), alcoholic hepatitis, insulin resistance in adipocytes, osteoarthritis, pancreatitis, metabolic syndrome, a trauma induced systemic inflammation, an acute respiratory distress syndrome (ARDS), a COVID-19-induced systemic inflammation, an organ rejection after transplantation, and others. See, for example, United States Patent Application Publication No. 2008/0311112 A1, published Dec. 18, 2008. The presently disclosed subject matter can also be used, in non-limiting embodiments, to treat post traumatic conditions, including ischemic injury and traumatic injury to the heart, liver, lung, kidney, intestine, brain, eye, and pancreas.

### 5.4 METHODS OF TREATMENT

In certain embodiments, the present disclosure provides for a method of treating an infectious or inflammatory disorder comprising administering, to a subject in need of such treatment, an effective amount of a compound of Formula I or Formula II that reduces one or more sign or symptom of inflammation in the subject.

In certain embodiments, the present disclosure provides for a method of treating an intestinal inflammatory disorder in a subject comprising administering, to a subject in need of such treatment, an effective amount of a compound of Formula I or Formula II that reduces intestinal inflammation in the subject. In certain particular embodiments, the intestinal inflammatory disorder is necrotizing enterocolitis. In certain embodiments, the present disclosure provides a method of treatment and/or prevention of necrotizing enterocolitis in premature infants.

In certain embodiments, the present disclosure provides for a method of treating an inflammatory pulmonary disease in a subject including administering, to a subject in need of such treatment, an effective amount of a compound of Formula I or Formula II that reduces pulmonary airway inflammation in the subject.

In certain embodiments, the present disclosure provides for a method of treating a traumatic injury in a subject including administering, to a subject in need of such treatment, an effective amount of a compound of Formula I or Formula II that reduces Tlr4-induced post-traumatic injury. In certain particular embodiments, the traumatic injury is to an organ selected from the group consisting of the heart, the liver, the lung, the kidney, the intestine, the brain, the eye, and the pancreas.

In certain embodiments, the present disclosure provides for a method of treating one or more of osteoarthritis, pancreatitis, metabolic syndrome, trauma induced systemic inflammation, acute respiratory distress syndrome (ARDS), COVID-19-induced systemic inflammation, organ rejection after transplantation, or other disorders of the gastrointestinal tract that can involve Tlr44 signaling such as e.g., ulcerative colitis or Crohn's disease.

In certain embodiments, the subject is a human or a non-human subject. In particular embodiments, the subject is a human subject. In certain embodiments, the subject is a non-human subject.

In certain embodiments, the compound of Formula I or Formula II can be administered by any standard route including but not limited to oral, intraperitoneal (i.p), intravenous (i.v.), subcutaneous (s.c.), intradermal, intramuscular (i.m.), intraarticular, intrathecal, intraarterial, intravaginal, rectal, nasal, and pulmonary.

In certain embodiments, an effective dose can be determined using methods known in the art (including, but not limited to, Tlr4 activity assays described herein). In certain embodiments, an effective dose can be between about 0.01 micromoles and about 50 micromoles of a compound of Formula I or Formula II per kilogram weight of the subject, or between about 0.1 micromoles and about 20 micromoles of a compound of Formula I or Formula II per kilogram weight of the subject.

In certain embodiments, the dose of a compound of Formula I or Formula II can be between about 0.001 milligrams and about 100 milligrams per kilogram weight of the subject, between about 0.01 milligrams and about 10 milligrams per kilogram weight of the subject, between about 0.1 milligrams and about 10 milligrams per kilogram weight of the subject, or between about 0.5 and 5 milligrams per kilogram weight of the subject.

### 6. EXAMPLES

The presently disclosed subject matter will be better understood by reference to the following Example, which is provided as exemplary of the presently disclosed subject matter, and not by way of limitation.

### Example 1: Exemplary Synthesis of Carbon Analogs of Pyranose Derivatives

The present Example provides synthesis of carbon analogs of pyranose derivatives according to certain embodiments of the present disclosure.

All reactions were performed under N₂ atmosphere that had been passed through a column of Drierite. All glassware were either flame-dried under high vacuum or dried in an oven overnight prior to use and allowed to cool under a stream of N₂. Reactions were stirred magnetically using Teflon-coated magnetic stirring bars, and syringe needles were dried in an oven and cooled in a desiccator cabinet over Drierite. The CH₂Cl₂ was freshly distilled over calcium hydride. All other materials were obtained from commercial sources and used as received. Reactions were monitored by thin-layer chromatography (TLC) analysis on pre-coated silica gel 60 F254 plates (250 μm layer thickness); visualization was accomplished by UV light (254 nm) and/or by staining with KMnO₄ solution (1.5 g KMnO₄ and 10 g K₂CO₃ in 200 mL H₂O with 10% NaOH, or PMA solution (10 g of phosphomolybdic acid in 100 mL of absolute ethanol). Flash chromatography was carried out on silica gel 60 (230-400 mesh). Melting points were determined in open capillary tubes, recorded on a Mel-Temp II apparatus fitted with a Fluke 51 II digital thermometer, and are uncorrected. Nuclear Magnetic Resonance (NMR) spectra were acquired on Bruker instruments operating at 300, 400, 500, and 600 MHz for 1H and ¹³C at ambient temperature. Chemical shifts (8) were reported in parts per million (ppm) with the residual solvent peak used as an internal standard (CDCl₃: 7.26 ppm for ¹H NMR and 77.16 ppm for ¹³C NMR). ¹H NMR were tabulated as follows: chemical shifts, multiplicity (s=singlet, d=doublet, t=triplet, m=multiplet, br=broad), coupling constant(s), and number of protons. High resolution mass spectra (HRMS) were obtained on a Thermo Scientific Exactive Orbitrap LC-MS (ESI positive ion mode) coupled to a Thermo Scientific Accela HPLC system using a 3.5 μM Water XTerra C18 column (2.1×50 mm; 10 min gradient elution with MeCN/H₂O/MeOH containing 0.1% formic acid at a flow rate of 500 μL/min from 3:92:5 at 0-0.5 min to 93:2:5 at 4.0, back to 3:92:5 from 6.0 to 7.5 min).

The present Example provides synthesis of (2R,3R,4R,5S,6R)-2-(acetoxymethyl)-6-propyltetrahydro-2H-pyran-3,4,5-triyl triacetate (7), (2R,3R,4R,5S,6R)-2-(acetoxymethyl)-6-isobutyltetrahydro-2H-pyran-3,4,5-triyl triacetate, (8) (2R,3S,4R,5R,6R)-2-isobutyl-6-((pivaloyloxy)methyl)tetrahydro-2H-pyran-3,4,5-triyl tris(2,2-dimethylpropanoate), (9) (2R,3R,4R,5S,6R)-2-(((3,3-dimethylbutanoyl)oxy)methyl)-6-isobutyltetrahydro-2H-pyran-3,4,5-triyl tris(3,3-dimethylbutanoate) (10) and (2R,3R,4R,5S,6R)-2-((benzoyloxy)methyl)-6-isobutyltetrahydro-2H-pyran-3,4,5-triyl tribenzoate (11) as shown by Scheme I below. Intermediates 3 and 4 were published by procedures previously reported in the literature.

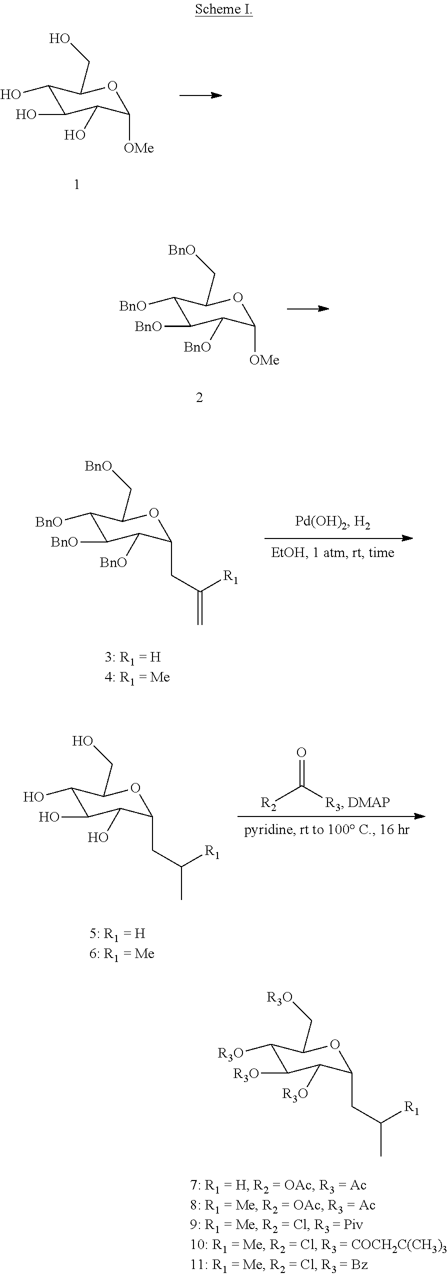

### Example 1.1—Synthesis of (2R,3S,4R,5R,6R)-2-(hydroxymethyl)-6-propyltetrahydro-2H-pyran-3,4,5-triol (5)

A suspension of 3 and 20% Pd(OH)₂/C in absolute EtOH is stirred under H₂ (balloon) at room temperature. The reaction mixture is filtered through celite, the filter pad is rinsed with EtOH, and the filtrate concentrated under reduced pressure and dried under vacuum to give crude 5, that was used in subsequent steps without further purification.

### Example 1.2—Synthesis of (2R,3S,4R,5R,6R)-2-(hydroxymethyl)-6-isobutyltetrahydro-2H-pyran-3,4,5-triol (6)

A suspension of 4 (550 mg, 0.950 mmol) and 20% Pd(OH)₂/C (123 mg) in absolute EtOH (12 mL) was stirred under H₂ (balloon) at room temperature for 16 hrs. The reaction mixture was filtered through celite, the filter pad was rinsed with EtOH and the filtrate concentrated under reduced pressure and dried under vacuum to give 209 mg of crude tetraol (100%, 0.96 mmol) as a colorless solid, that was used in subsequent steps without further purification. ¹H NMR (MeOD, 500 MHz) δ 4.02-3.98 (m, 1H), 3.76 (dd, J=11.7, 2.3 Hz, 1H), 3.59 (dd, J=11.7, 1.0 Hz, 1H), 3.51 (t, J=8.7 Hz, 1H), 3.42-3.38 (m, 1H), 3.25 (t, J=8.7 Hz, 1H), 1.82-1.78 (m, 1H), 1.72-1.66 (m, 1H), 1.38-1.33 (m, 1H), 1.18 (t, J=7.0 Hz, 1H), 0.97 (d, J=6.7 Hz, 3H), 0.93 (d, J=6.5 Hz, 3H).

### Example 1.3—Synthesis of (2R,3R,4R,5S,6R)-2-(acetoxymethyl)-6-propyltetrahydro-2H-pyran-3,4,5-triyl triacetate (7)

To a suspension of 5 (100 mg, 0.49 mmol) and acetic anhydride (0.23 mL, 2.4 mmol) in pyridine (0.2 mL), was added a catalytic amount of DMAP. The solution was stirred at room temperature for 16 hours and diluted with ethyl acetate (10 mL). The organic phase was washed sequentially with 1 M HCl (3×5 mL), water (1×5 mL), saturated aq NaHCO₃ (1×5 mL) and brine (5 mL×1). The resulting organic phase was dried (Na₂SO₄), concentrated and the resulting residue dried under vacuum to give a 4:1 ratio of α and β pentaacetylated anomers. Recrystallization from a mixture of Hexanes/EtOAc gave 137 mg of 7 (75%, 0.366 mmol) as white needles. M.P.; 102-103.5° C. ¹H NMR (CDCl₃, 500 MHz) δ 5.32 (t, J=9.3 Hz, 1H), 5.07 (dd, J=9.3, 5.8 Hz, 1H), 4.99 (t, J=9.3 Hz, 1H), 4.24 (dd, J=12.2, 5.3 Hz, 1H), 4.20-4.16 (m, 1H), 4.08 (dd, J=12.2, 2.5 Hz, 1H), 3.83-3.80 (m, 1H), 2.09 (s, 3H), 2.05 (s, 3H), 2.03 (s, 3H), 2.03 (s, 3H), 1.81-1.74 (m, 1H), 1.50-1.41 (m, 2H), 1.35-1.29 (m, 1H), 0.97 (t, J=7.1 Hz, 3H). ¹³C NMR (CDCl₃, 500 MHz) δ 170.8, 170.4, 169.8, 169.7, 72.6, 70.7, 69.1, 68.7, 62.5, 27.5, 20.9, 20.8, 18.4, 13.9. HRMS Calcd. For C₁₇H₂₇O₉ [M+H]⁺: 375.1650; found: 375.1647.

### Example 1.4—Synthesis of (2R,3R,4R,5S,6R)-2-(acetoxymethyl)-6-isobutyltetrahydro-2H-pyran-3,4,5-triyl triacetate (8)

To a solution of 6 (42 mg, 0.19 mmol) and a catalytic amount of DMAP in pyridine (0.65 mL), was added acetic anhydride (0.18 mL, 1.91 mmol). The reaction mixture was stirred at room temperature for 16 hours and diluted with EtOAc (5 mL). The organic phase was washed sequentially with 1M HCl (5 mL×2), sat aq NaHCO₃ (5 mL×2), and brine (5 mL×1). The organic phase was dried (Na₂SO₄) and concentrated under reduced pressure. The resulting residue was purified on SiO₂ (Hexanes/EtOAc, 8:1) to give 50 mg of 8 (76%, 0.129 mmol) as an amorphous solid. M.P.; 58.5-60° C. ¹H NMR (CDCl₃, 400 MHz) δ 5.31 (t, J=9.2 Hz, 1H), 5.06 (dd, J=9.6, 5.8 Hz, 1H), 4.99 (t, J=9.2 Hz, 1H), 4.30-4.24 (m, 1H), 4.24 (dd, J=12.1, 5.1 Hz, 1H), 4.06 (dd, J=12.1, 2.6 Hz, 1H), 3.84-3.80 (m, 1H), 2.08 (s, 3H), 2.05 (s, 3H), 2.03 (s, 3H), 2.02 (s, 3H), 1.82-1.78 (m, 2H), 1.27-1.18 (m, 1H), 0.97 (d, J=6.6 Hz, 3H), 0.91 (d, J=6.5 Hz, 3H). ¹³C NMR (CDCl₃, 400 MHz) δ 170.8, 170.4, 169.8, 169.7, 71.0, 70.7, 70.6, 69.1, 68.8, 62.5, 33.8, 24.2, 23.6, 21.5, 20.9. HRMS Calcd. For C₁₈H₂₉O₉ [M+H]⁺: 389.1806; found: 389.1801.

### Example 1.5—Synthesis of (2R,3S,4R,5R,6R)-2-isobutyl-6-((pivaloyloxy)methyl)tetrahydro-2H-pyran-3,4,5-triyl tris(2,2-dimethylpropanoate) (9)

To a solution of 6 (50 mg, 0.23 mmol) and a catalytic amount of DMAP in pyridine (0.8 mL), was added pivaloyl chloride (0.28 mL, 2.23 mmol). The reaction mixture was stirred at room temperature for 16 hours and diluted with EtOAc (5 mL). The organic phase was washed sequentially with 1M HCl (5 mL×2), sat aq NaHCO₃ (5 mL×2), and brine (5 mL×1). The organic phase was dried (Na₂SO₄) and concentrated under reduced pressure. The resulting residue was purified on SiO₂ (Hexanes/EtOAc, 18:1) to give 81 mg of 9 (64%, 0.146 mmol) as an amorphous solid. M.P.; 123-124° C. ¹H NMR (CDCl₃, 600 MHz) δ 5.41 (t, J=9.7 Hz, 1H), 5.06 (dd, J=10.0 Hz, 1H), 5.01 (t, J=9.7 Hz, 1H), 4.29-4.25 (m, 1H), 4.12 (dd, J=12.0, 1.2 Hz, 1H), 4.00 (dd, J=12.0, 6.4 Hz, 1H), 3.82-3.80 (m, 1H), 1.88-1.83 (m, 1H), 1.76-1.71 (m, 1H), 1.21 (s, 9H), 1.17 (s, 9H), 1.16 (s, 9H), 1.12 (s, 9H), 0.98 (d, J=6.7 Hz, 3H), 0.88 (d, J=6.5 Hz, 3H). ¹³C NMR (CDCl₃, 600 MHz) δ 178.3, 177.3, 177.2, 176.8, 71.0, 70.9, 70.2, 68.9, 63.0, 39.0, 38.9 (2 signals), 33.4, 27.3 (2 signals), 27.2, 23.8 (2 signals), 21.3. HRMS Calcd. For C₃₀H₅₃O₉ [M+H]⁺: 557.3684; found: 557.3680.

### Example 1.6—Synthesis of (2R,3R,4R,5S,6R)-2-(((3,3-dimethylbutanoyl)oxy)methyl)-6-isobutyltetrahydro-2H-pyran-3,4,5-triyl tris(3,3-dimethylbutanoate) (10)

To a solution of 6 (50 mg, 0.23 mmol) and catalytic amount of DMAP in pyridine (0.8 mL), was added 3,3-Dimethylbutyryl chloride (0.32 mL, 2.27 mmol). The reaction mixture was stirred at room temperature for 16 hours and diluted with EtOAc (5 mL). The organic phase was washed sequentially with 1M HCl (5 mL×2), sat aq NaHCO₃ (5 mL×2), and brine (5 mL×1). The organic phase was dried (Na₂SO₄) and concentrated under reduced pressure. The resulting residue was purified on SiO₂ (Hexanes/EtOAc, 20:1) to give 118 mg of 10 (85%, 0.146 mmol) as an amorphous solid. M.P.; 83-84° C. ¹H NMR (CDCl₃, 500 MHz) δ 5.37 (t, J=9.3 Hz, 1H), 5.03 (dd, J=9.7 Hz, 1H), 4.98 (t, J=9.1 Hz, 1H), 4.29-4.25 (m, 1H), 4.15-4.09 (m, 2H), 3.84-3.80 (m, 1H), 2.23 (s, 2H), 2.22-2.11 (m, 4H), 2.15 (s, 2H), 1.82-1.75 (m, 1H), 1.74-1.69 (m, 1H), 1.24-1.19 (m, 1H), 1.02 (s, 9H), 1.01 (s, 9H), 1.00 (s, 9H), 0.98 (s, 1H), 0.95 (d, J=6.7 Hz, 3H), 0.88 (d, J=6.5 Hz, 3H). ¹³C NMR (CDCl₃, 500 MHz) δ 172.0, 171.3, 171.1, 170.9, 70.9, 70.7, 69.8, 69.2, 69.0, 62.5, 47.8 (2 signals), 47.7, 47.6, 33.9, 30.9, 30.8, 30.7, 30.6, 29.7 (3 signals), 24.1, 23.7, 21.5. HRMS Calcd. For C₃₄H₆₁₀₉ [M+H]⁺: 613.4310; found: 613.4307.

### Example 1.7—Synthesis of (2R,3R,4R,5S,6R)-2-((benzoyloxy)methyl)-6-isobutyltetrahydro-2H-pyran-3,4,5-triyl tribenzoate (11)

To a solution of 6 (34 mg, 0.154 mmol) and catalytic amount of DMAP in pyridine (0.8 mL), was added benzoyl chloride (0.18 mL, 1.54 mmol). The reaction mixture was stirred at room temperature for 16 hours and diluted with EtOAc (5 mL). The organic phase was washed sequentially with 1M HCl (5 mL×2), sat aq NaHCO₃ (5 mL×2), and brine (5 mL×1). The organic phase was dried (Na₂SO₄) and concentrated under reduced pressure. The resulting residue was purified on SiO₂ (Hexanes/EtOAc, 18:1) to give 73 mg of 11 (82%, 0.127 mmol) as a white crystalline solid. M.P.; 129.5-131° C. ¹H NMR (CDCl₃, 500 MHz) δ 8.04 (d, J=7.2 Hz, 2H), 7.98 (d, J=7.3 Hz, 2H), 7.93 (d, J=7.3 Hz, 2H), 7.90 (d, J=7.3 Hz, 2H), 7.57-7.38 (m, 8H), 7.35-7.31 (m, 4H), 5.99 (t, J=9.0 Hz, 1H), 5.57 (t, J=9.0 Hz, 1H), 5.50 (dd, J=9.0, 5.7 Hz, 1H), 4.61 (m, 1H), 4.55 (d, J=4.7 Hz, 1H), 4.31-4.28 (m, 1H), 2.09-2.03 (m, 1H), 1.87-1.81 (m, 1H), 1.40-1.35 (m, 1H), 0.99 (d, J=6.7 Hz, 3H), 0.90 (d, J=6.5 Hz, 3H). ¹³C NMR (CDCl₃, 500 MHz) δ 166.4, 166.0, 165.5 (2 signals), 133.6, 133.5, 133.4, 133.2, 130.0 (2 signals), 129.9, 129.3, 129.2, 129.1, 128.6, 71.6, 71.0, 70.7, 70.0, 69.7, 63.5, 34.4, 24.3, 23.7, 21.5. HRMS Calcd. For C₃₈H₃₇O₉ [M+NH₄]⁺: 654.2698; found: 654.2684.

The present Example further provides synthesis of (2R,3R,4R,5S,6R)-2-(acetoxymethyl)-6-isobutyl-5-(pivaloyloxy)tetrahydro-2H-pyran-3,4-diyl acetate (14) and (2R,3R,4R,5S,6R)-2-(acetoxymethyl)-5-((3,3-dimethylbutanoyl)oxy)-6-isobutyltetrahydro-2H pyran-3,4-diyl diacetate (15) as shown by Scheme II.

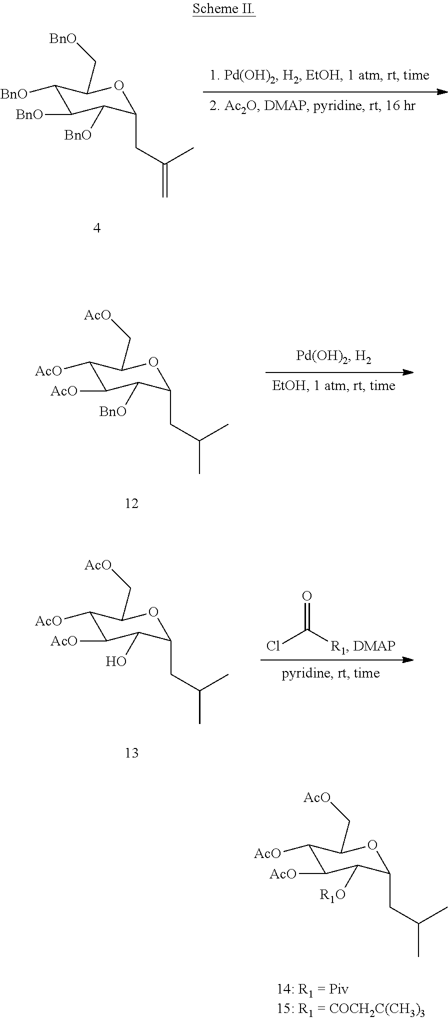

### Example 1.8—Synthesis of (2R,3R,4R,5S,6R)-2-(acetoxymethyl)-5-(benzyloxy)-6-isobutyltetrahydro-2H-pyran-3,4-diyl diacetate (12)

To a suspension of 4 (787 mg, 1.36 mmol) and 20% Pd(OH)₂/C (176 mg) in EtOH (17 mL) was stirred under H₂ (balloon) at room temperature for 16 hours. The reaction mixture was passed through Celite, the filter pad rinsed with EtOH and the filtrate concentrated and dried under vacuum to give 348 mg of a 2:1 mixture of tetraol and triol as a colorless solid. To a solution of the crude intermediate and a catalytic amount of DMAP was added acetic anhydride (1.3 mL, 13.6 mmol) dropwise. The reaction mixture was stirred at room temperature for 16 hours and diluted with EtOAc (10 mL). The organic phase was washed sequentially with 1M HCl (10 mL×2), sat aq NaHCO₃ (10 mL×2), and brine (10 mL×2 mL). The organic phase was dried (Na₂SO₄) and concentrated under reduced pressure. The resulting residue was purified on SiO₂ (Hexanes/EtOAc, 4:1) to give 165 mg of 12 (28%, 0.378 mmol) as a white crystalline solid. ¹H NMR (CDCl₃, 500 MHZ) δ 7.25-7.26 (m, 4H), 5.28 (t, J=9.5 Hz, 1H), 4.91 (t, J=9.5 Hz, 1H), 4.56 (d, J=3.9 Hz, 2H), 4.25 (dd, J=12.2, 4.9 Hz, 1H), 4.12-4.09 (m, 1H), 4.00 (dd, J=12.2, 2.3 Hz, 1H), 3.80-3.77 (m, 1H), 3.68 (dd, J=9.7, 5.9 Hz), 2.06 (s, 3H), 2.01 (s, 6H), 1.80-1.65 (m, 2H), 1.43-1.40 (m, 1H), 0.96 (d, J=6.7 Hz, 3H), 0.89 (d, J=6.5 Hz, 3H). 13C NMR (CDCl₃, 500 MHz) & 170.9, 170.5, 170.0, 137.9, 128.6, 128.1, 127.9, 73.2, 72.5, 72.4, 69.5, 68.4, 62.7, 33.0, 24.3, 23.7, 21.5, 21.0, 20.9, 20.8.

### Example 1.9—Synthesis of (2R,3R,4R,5S,6R)-2-(acetoxymethyl)-5-hydroxy-6-isobutyltetrahydro-2H-pyran-3,4-diyl diacetate (13)

To a solution of 12 (140 mg, mmol) and 20% Pd(OH)₂/C (31 mg) in EtOH (4 mL), was stirred under H₂ (balloon) at room temperature for 16 hours. The reaction mixture was passed through Celite, the filter pad rinsed with EtOH and the filtrate concentrated under reduced pressure to give 100 mg of 13 (90%, 0.29 mmol) as a colorless solid. M.P.; 73-76° C. ¹H NMR (CDCl₃, 400 MHz) δ 5.10 (t, J=8.4 Hz, 1H), 4.94 (t, J=8.4 Hz, 1H), 4.31 (dd, J=12.1, 5.6 Hz, 1H), 4.17-4.12 (m, 1H), 4.07 (dd, J=12.1, 5.6 Hz, 1H), 389-3.83 (m, 1H), 2.23 (d, J=6.6 Hz, 1H), 2.10 (s, 3H), 2.08 (s, 3H), 2.05 (s, 3H), 1.77-1.68 (m, 2H), 1.47-1.40 (m, 1H), 0.99 (d, J=6.5, 3H), 0.95 (d, J=6.5 Hz, 3H). ¹³C NMR (CDCl₃, 500 MHz) δ171.4, 170.9, 169.7, 73.7, 72.9, 70.2, 69.5, 68.7, 62.4, 33.7, 24.4, 23.7, 21.7, 21.1, 20.9 (2 signals). HRMS Calcd. For C₁₆H₂₇O₈ [M+H]⁺: 347.1700; found: 347.1698.

### Example 1.10—(2R,3R,4R,5S,6R)-2-(acetoxymethyl)-6-isobutyl-5-(pivaloyloxy)tetrahydro-2H-pyran-3,4-diyl diacetate (14)

To a solution of 13 (48 mg, 0.14 mmol), pyridine (0.1 mL) and a catalytic amount of DMAP in anhydrous CH₂Cl₂ (1.5 mL) at room temperature, was added pivaloyl chloride (0.042 mL, 0.346 mmol). The reaction mixture was stirred for 16 hours and diluted with CH₂Cl₂ (5 mL). The organic phase was washed sequentially with 1 M aq HCl (5 mL×2), sat aq NaHCO₃ (5 mL×2), and brine (5 mL×1). The organic phase was dried (Na₂SO₄) and concentrating under reduced pressure. The resulting residue was purified on SiO₂ (Hexanes/EtOAc, 8:1) to give 19 mg of 14 (32%, 0.044 mmol) as a colorless amorphous solid. ¹H NMR (CDCl₃, 600 MHZ) δ 5.35 (t, J=9.3 Hz, 1H), 5.02 (dd, J=10.1 Hz, 1H), 5.00 (t, J=9.3 Hz, 1H), 4.30-4.28 (m, 1H), 4.26 (dd, J=12.2, 5.2 Hz, 1H), 4.06 (dd, J=12.2, 2.4 Hz, 1H), 3.83 (m, 1H), 2.09 (s, 1H), 2.03 (s, 1H), 2.00 (s, 1H), 1.82-1.77 (m, 1H), 1.73-1.68 (m, 1H), 1.19-1.14 (m, 1H), 0.97 (d, J=6.7 Hz, 3H), 0.90 (d, J=6.5 Hz, 3H). ¹³C NMR (CDCl₃, 600 MHz) δ 177.2, 170.9, 170.3, 169.8, 71.3, 70.5, 70.4, 69.1, 68.8, 62.6, 38.9, 33.6, 27.1, 24.3, 23.7, 21.6, 20.9, 20.8. HRMS Calcd. For C₂₁H₃₅O₉ [M+H]⁺: 431.2276; found: 431.2269.

### Example 1.11 (2R,3R,4R,5S,6R)-2-(acetoxymethyl)-5-((3,3-dimethylbutanoyl)oxy)-6-isobutyltetrahydro-2H-pyran-3,4-diyl diacetate (15)

To a solution of 13 (48 mg, 0.14 mmol), pyridine (0.1 mL) and a catalytic amount of DMAP in anhydrous CH₂Cl₂ (1.5 mL) at room temperature, was added 3,3-Dimethylbutyryl chloride (0.05 mL, 0.35 mmol). The reaction mixture was stirred for 16 hours and diluted with CH₂Cl₂ (5 mL). The organic phase was washed sequentially with 1 M aq HCl (5 mL×2), sat aq. NaHCO₃ (5 mL×2), and brine (5 mL×1). The organic phase was dried (Na₂SO₄) and concentrating under reduced pressure. The resulting residue was purified on SiO₂ (Hexanes/EtOAc, 9:1) to give 29 mg of 15 (47%, 0.065 mmol) as a colorless amorphous solid. ¹H NMR (CDCl₃, 600 MHz) δ 5.34 (t, J=9.5 Hz, 1H), 5.08 (dd, J=9.5, 5.9 Hz, 1H), 4.98 (t, J=9.5 Hz, 1H), 4.32-4.28 (m, 1H), 4.24 (dd, J=12.2, 5.0 Hz, 1H), 4.06 (dd, J=12.2, 2.3 Hz, 1H), 3.84-3.81 (m, 1H), 2.19 (s, 2H), 2.08 (s, 3H), 2.03 (s, 3H), 2.00 (s, 3H), 1.81-1.76 (m, 1H), 1.72-1.68 (m, 1H), 1.27-1.19 (m, 1H), 1.00 (s, 9H), 0.97 (d, J=6.7 Hz, 3H), 0.90 (d, J=6.5 Hz, 3H). ¹³C NMR (CDCl₃, 600 MHZ) δ 171.1, 170.8, 170.4, 169.8, 71.2, 70.6, 70.3, 69.4, 68.6, 62.5, 47.8, 33.6, 30.9, 29.6, 24.2, 23.7, 21.5, 20.9 (2 signals), 20.8. HRMS Calcd. For C₂₂H₃₇O₉ [M+H]⁺: 445.2432; found: 445.2425.

The present Example further provides a suggested synthesis of (2R,3S,4R,5S,6R)-2-(acetoxymethyl)-6-isobutyl-5-(pivaloyloxy)tetrahydro-2H-pyran-3,4-diyl diacetate (21) as shown by Scheme III.

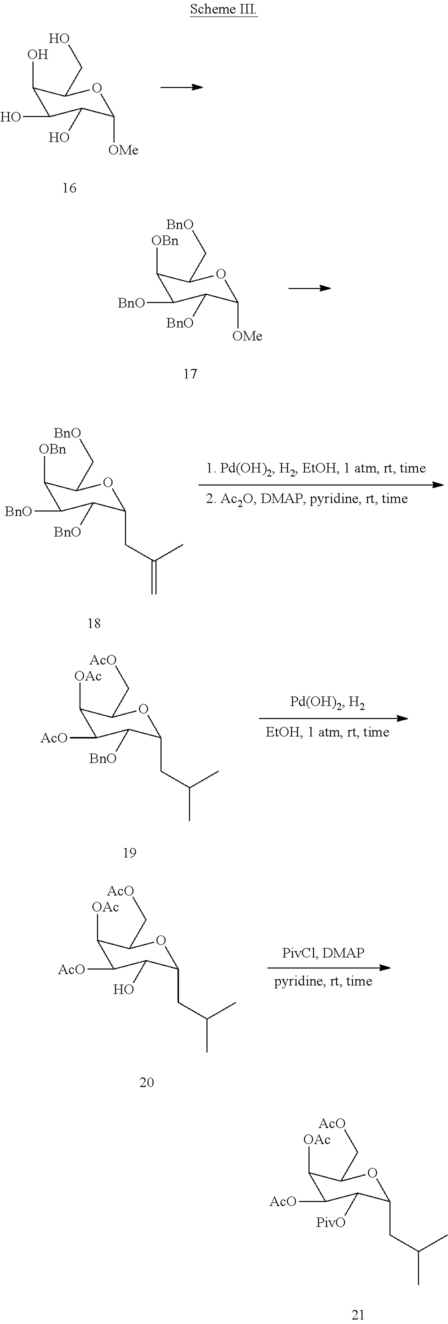

### Example 1.12—Synthesis of (2R,3S,4R,5S,6R)-2-(acetoxymethyl)-5-(benzyloxy)-6-isobutyltetrahydro-2H-pyran-3,4-diyl diacetate (19)

A suspension of 18 and 20% Pd(OH)₂/C in EtOH is stirred under H₂ (balloon) at room temperature. The reaction mixture is passed through Celite, the filter pad rinsed with EtOH and the filtrate concentrated and dried under vacuum. To a solution of the crude intermediate and a catalytic amount of DMAP is added acetic anhydride dropwise. The reaction mixture is stirred at room temperature and diluted with EtOAc. The organic phase is washed sequentially with 1M HCl, sat aq NaHCO₃, and brine. The organic phase is dried (Na₂SO₄) and concentrated under reduced pressure to give 19 (See, e.g., Cipolla, L.; Lay, L.; Nicotra, F. J. Org. Chem. 1997, 62 (19), 6678-6681.).

### Example 1.13—Synthesis of (2R,3S,4R,5S,6R)-2-(acetoxymethyl)-5-hydroxy-6-isobutyltetrahydro-2H-pyran-3,4-diyl diacetate (20)

A solution of 19 and 20% Pd(OH)₂/C in EtOH, is stirred under H₂ (balloon) at room temperature. The reaction mixture is passed through Celite, the filter pad rinsed with EtOH, and the filtrate concentrated under reduced pressure to give 20.

### Example 1.14—(2R,3S,4R,5S,6R)-2-(acetoxymethyl)-6-isobutyl-5-(pivaloyloxy)tetrahydro-2H-pyran-3,4-diyl diacetate (21)

To a solution of 20, pyridine, and a catalytic amount of DMAP in anhydrous CH₂Cl₂ at room temperature, is added pivaloyl chloride. The reaction mixture is stirred and then diluted with CH₂Cl₂. The organic phase is washed sequentially with 1 M aq HCl, sat aq. NaHCO₃, and brine. The organic phase is dried (Na₂SO₄) and concentrating under reduced pressure. The resulting residue is purified on SiO₂ to give 21.

The present Example further provides a suggested synthesis of (2R,3S,4R,5R,6S)-5-acetamido-2-(acetoxymethyl)-6-(prop-2-yn-1-yloxy)tetrahydro-2H-pyran-3,4-diyl diacetate (24) as shown by Scheme IV.

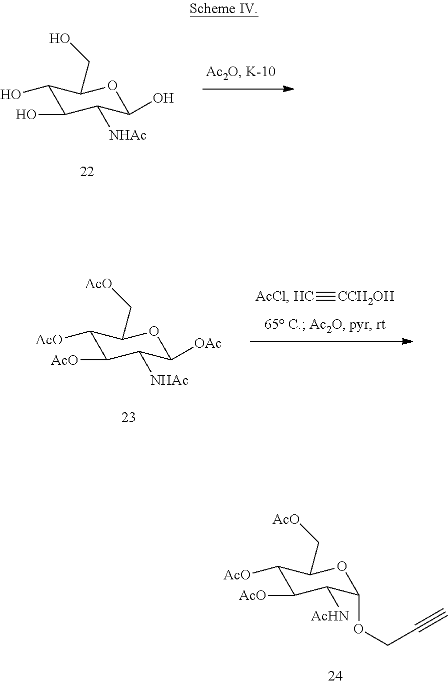

### Example 1.15—Synthesis of (2R,3S,4R,5R,6S)-5-acetamido-2-(acetoxymethyl)-6-(prop-2-yn-1-yloxy)tetrahydro-2H-pyran-3,4-diyl diacetate (24)

A suspension of 22, acetic anhydride, and montmorillonite K-10 is stirred to give 23. Then, a solution of 23, in situ prepared 5% HCl, and propargyl alcohol is stirred at 65° C. To the solution is added acetic anhydride and pyridine. The solution is then stirred at room temperature to give 24 (See, e.g., Wipf, P.; Eyer, B. R.; Yamaguchi, Y.; Zhang, F.; Neal, M. D.; Sodhi, C. P.; Good, M.; Branca, M.; Prindle, T., Jr.; Lu, P.; Brodsky, J. L.; Hackam, D. J. Tetrahedron Letters. 2015, 56 (23), 3097-3100).

The present Example further provides synthesis of (2R,3S,4R,5S,6R)-2-(acetoxymethyl)-5-pivalamido-6-propyltetrahydro-2H-pyran-3,4-diyl diacetate (28) as shown by Scheme V.

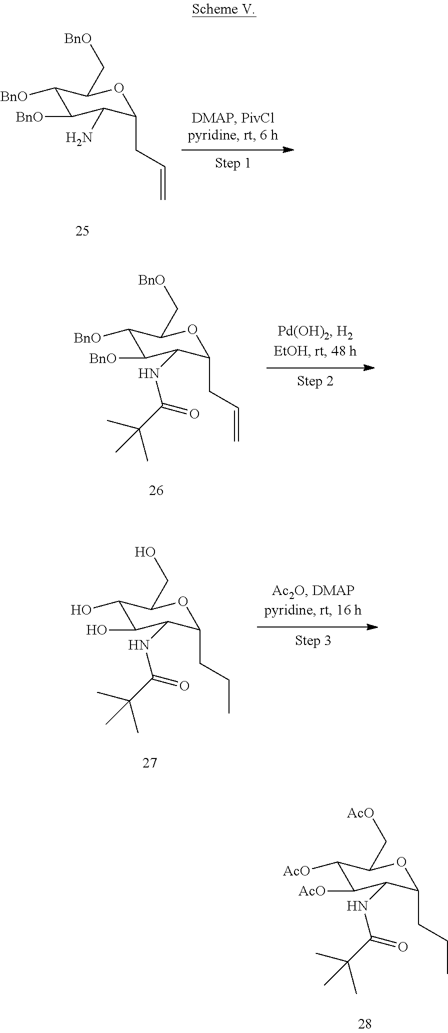 [SMILES: C=CCC1OC(COCc2ccccc2)C(OCc2ccccc2)C(OCc2ccccc2)C1N ; C=CCC1OC(COCc2ccccc2)C(OCc2ccccc2)C(OCc2ccccc2)C1NC(=O)C(C)(C)C ; CCCC1OC(CO)C(O)C(O)C1NC(=O)C(C)(C)C ; CCCC1OC(COC(C)=O)C(OC(C)=O)C(OC(C)=O)C1NC(=O)C(C)(C)C]

### Example 1.16—N-((2R,3S,4R,5S,6R)-2-Allyl-4,5-bis(benzyloxy)-6-((benzyloxy)methyl)tetrahydro-2H-pyran-3-yl)pivalamide (26)

To a solution of 25 (265 mg, 0.560 mmol) and a catalytic amount of DMAP in an 8:1 mixture of CH₂Cl₂ and pyridine (3 mL) at room temperature was added pivaloyl chloride (0.082 mL, 0.67 mmol) in one portion. After 6 h, the reaction mixture was concentrated under reduced pressure and the resulting residue purified by chromatography on SiO₂ (Hexanes/EtOAc, 4:1) to give 26 (194 mg, 62%) as a colorless solid: Mp 32-34° C.; HRMS m/z calcd for C₃₅H₄₄O₅N ([M+1]⁺) 558.3214, found 558.3213; ¹H NMR (500 MHZ, CDCl₃) δ 7.35-7.20 (m, 15H), 6.79 (d, J=9.5 Hz), 5.83-5.78 (m, 1H), 5.10-5.02 (m, 2H), 4.63-4.57 (m, 2H), 4.52 (s, 2H), 4.49-4.43 (m, 2H), 4.34-4.32 (m, 1H), 3.95-3.93 (m, 1H), 3.88 (dd, J=7.0, 10.0 Hz), 3.73 (dd, J=7.0, 10.0 Hz), 3.65 (bs, 1H), 3.58-3.57 (m, 1H), 2.25-2.10 (m, 1H), 1.04 (s, 9H).

### Example 1.17—N-((2R,3R,4R,5S,6R)-4,5-Dihydroxy-6-(hydroxymethyl)-2-propyltetrahydro-2H-pyran-3-yl)pivalamide (27)

A slurry of 26 (134 mg, 1.14 mmol) and 20% Pd(OH)₂/C (30 mg) in EtOH (3.5 mL) was stirred under H₂ (100 psi) in a Parr reactor at room temperature for 48 h. The reaction mixture was passed through Celite® and the filtrate concentrated under reduced pressure to give 27 (66 mg, 0.23 mmol, 96%,) as a colorless solid that was used in subsequent steps without further purification.

### Example 1.18—(2R,3S,4R,5S,6R)-2-(Acetoxymethyl)-5-pivalamido-6-propyltetrahydro-2H-pyran-3,4-diyl diacetate (28)

To a solution of 27 (32 mg, 0.11 mmol) and acetic anhydride (0.08 mL, 0.83 mmol) in pyridine (0.65 mL) was added a catalytic amount of DMAP. The solution was stirred at room temperature for 16 h, and diluted with ethyl acetate (7.5 mL). The organic phase was washed sequentially with 1 M HCl (3×5 mL), water (1×5 mL), saturated aq NaHCO₃ (1×5 mL), brine (5 mL×1), then dried (Na₂SO₄) and concentrated under reduced pressure. The resulting residue was purified by chromatography on SiO₂ (Hexanes/EtOAc, 4:1) to give 28 (34 mg, 0.082 mmol, 74%) as a colorless, amorphous solid: HRMS m/z calcd for C₂₀H₃₄₀₈N ([M+1]⁺) 416.2279, found 416.2309; ¹H NMR (400 MHZ, CDCl₃) δ 6.06 (d, J=8.0 Hz, 1H), 5.05-5.01 (m, 1H), 4.98-4.94 (m, 1H), 4.36 (dd, J=12.0, 6.4 Hz, 1H), 4.24-4.20 (m, 1H), 4.17-4.01 (m, 2H), 3.86-3.84 (m, 1H), 2.09 (s, 3H), 2.08 (s, 3H), 2.07 (s, 3H), 1.65-1.55 (m, 2H), 1.35-1.25 (m, 2H), 1.18 (s, 9H), 0.93 (t, J=7.2 Hz, 3H).

The present Example further provides a suggested synthesis of (2R,3S,4R,5S,6R)-6-allyl-2-((butyryloxy)methyl)-5-pivalamidotetrahydro-2H-pyran-3,4-diyl dibutyrate (31), (2R,3S,4R,5S,6R)-2-(hydroxymethyl)-6-isobutyl-5-pivalamidotetrahydro-2H-pyran-3,4-diyl diacetate (32), (1S,2S,4S,6S)-2-acetoxy-4-(acetoxymethyl)-6-propoxycyclohexyl pivalate (33), N-((2S,3R,4R,5S,6R)-4,5-dihydroxy-6-(hydroxymethyl)-2-isopropyltetrahydro-2H-pyran-3-yl)pivalamide (34), (2R,3S,4R,5S,6R)-6-allyl-2-((benzoyloxy)methyl)-4-(2-phenylacetoxy)-5-pivalamidotetrahydro-2H-pyran-3-yl benzoate (35), and (2R,3S,4R,5S,6R)-2-(((azetidine-1-carbonyl)oxy)methyl)-5-pivalamido-6-propyltetrahydro-2H-pyran-3,4-diyl bis(azetidine-1-carboxylate) (36) as shown by Scheme VI.

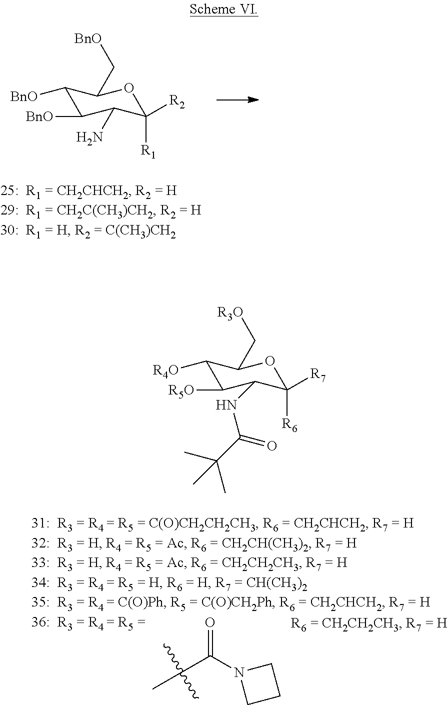

### Example 1.19—(2R,3S,4R,5S,6R)-6-allyl-2-((butyryloxy)methyl)-5-pivalamidotetrahydro-2H-pyran-3,4-diyl dibutyrate (31)

Starting with 25, a synthesis analogous to Scheme V is used to give 31. The alkene can be retained in the debenzylation step using deprotection conditions (See, e.g., Cavedon, C.; Sletten, E. T.; Madani, A.; Niemeyer, O.; Seeberger, P. H.; Pieber, B. Org. Lett. 2021, 23 (2), 514-518).

### Example 1.20—(2R,3S,4R,5S,6R)-2-(hydroxymethyl)-6-isobutyl-5-pivalamidotetrahydro-2H-pyran-3,4-diyl diacetate (32)

Starting with 29, a synthesis analogous to Scheme V is used. Further steps using the product of Step 3 can achieved with selective deprotection at the C-6 hydroxyl using the methods such as those disclosed in Filice, et al. (See, Filice, M.; Guisan, J. M.; Terreni, M.; Palomo, J. M. Nature Protocols. 2012, 7 (10), 1783-1796.) to give 32.

### Example 1.21—(2R,3S,4R,5S,6R)-2-(hydroxymethyl)-5-pivalamido-6-propyltetrahydro-2H-pyran-3,4-diyl diacetate (33)

Starting with 25, a synthesis analogous to Scheme V is used. Further steps using the product of Step 3 can achieved with selective deprotection at the C-6 hydroxyl using the methods such as those disclosed in Filice, et al. (See, Filice, M.; Guisan, J. M.; Terreni, M.; Palomo, J. M. Nature Protocols. 2012, 7 (10), 1783-1796.) to give 33.

### Example 1.22—N-((2S,3R,4R,5S,6R)-4,5-dihydroxy-6-(hydroxymethyl)-2-isopropyltetrahydro-2H-pyran-3-yl)pivalamide (34)

Starting with 30 a synthesis analogous to Steps 1-2 of Scheme V is used to give 34 (See, e.g., Wipf, P.; Pierce, J. G.; Zhuang, N. Org. Lett. 2005, 7 (3), 483-485.).

### Example 1.23—(2R,3S,4R,5S,6R)-6-allyl-2-((benzoyloxy)methyl)-4-(2-phenylacetoxy)-5-pivalamidotetrahydro-2H-pyran-3-yl benzoate (35)

Starting with 25, a synthesis analogous to Scheme V is used. The alkene can be retained in the debenzylation step using deprotection conditions (See, e.g., Cavedon, C.; Sletten, E. T.; Madani, A.; Niemeyer, O.; Seeberger, P. H.; Pieber, B. Org. Lett. 2021, 23 (2), 514-518). Further steps using the product of Step 3 can achieved with selective deprotection followed by selective protection at the C-6 hydroxyl followed at the C-6 hydroxyl using the methods such as those disclosed in Filice, et al. (See, Filice, M.; Guisan, J. M.; Terreni, M.; Palomo, J. M. Nature Protocols. 2012, 7 (10), 1783-1796.) to give 35.

### Example 1.24—(2R,3S,4R,5S,6R)-2-(((azetidine-1-carbonyl)oxy)methyl)-5-pivalamido-6-propyltetrahydro-2H-pyran-3,4-diyl bis(azetidine-1-carboxylate) (36)

Starting with 25, a synthesis analogous to Scheme V is used. Step 3 is accomplished using 1-azetidinecarbonyl chloride using methods such as those disclosed in WO 2017/023631 A1 to give 36.

The present Example further provides synthesis of (1R,2S,3S)-5-(((3,3-Dimethylbutanoyl)oxy)methyl)-3-propoxycyclohexane-1,2-diyl bis(3,3-dimethylbutanoate) (42) as shown by Scheme VII.

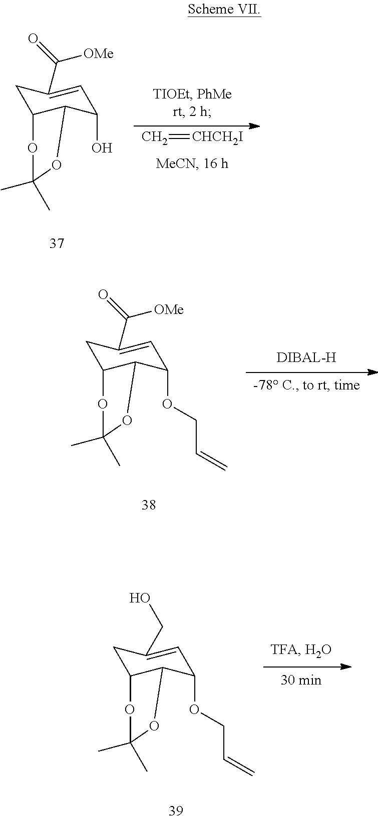

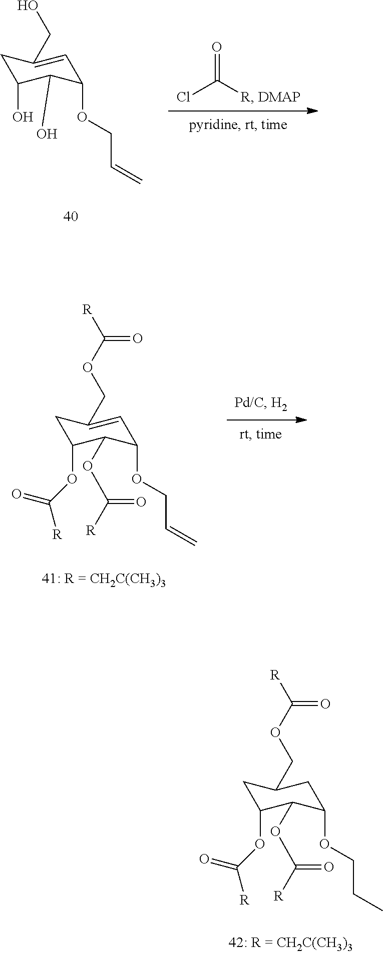

### Example 1.25—Methyl (3aR,7S,7aR)-7-(allyloxy)-2,2-dimethyl-3a,4,7,7a-tetrahydrobenzo[d][1,3]dioxole-5-carboxylate (38)

To a solution of 37 (230 mg, 1.01 mmol) in toluene (3.6 mL) at room temperature was added TlOEt (0.1 mL). After 2 h, the reaction mixture was concentrated under reduced pressure, and a solution of the residue in MeCN (3.6 mL) was treated with allyl iodide (0.3 mL) and stirred in the dark. After 16 h, the solids were removed by filtering through Celite®, the filter pad was rinsed with CH₂Cl₂, the filtrate was concentrated under reduced pressure and the residue was purified by chromatography on SiO₂ (Hexanes/EtOAc, 2:3) to give 46 mg of 38 as an orange oil: ¹H NMR (CDCl₃, 400 MHZ) δ 7.04 (s, 1H), 6.01-5.91 (m, 1H), 5.34-5.29 (m, 1H), 5.25-5.22 (dd, J=10.4, 1.2 Hz, 1H), 4.67-4.64 (m, 1H), 4.62-4.59 (m, 1H), 4.30-4.25 (m, 1H), 4.20-4.14 (m, 1H), 3.87-3.85 (m, 1H), 3.77 (s, 3H), 3.03 (d, J=16.3 Hz, 1H), 1.94-1.84 (m, 1H), 1.32 (s, 3H), 1.31 (s, 3H); ¹³C NMR (CDCl₃, 400 MHZ) δ 166.2, 140.8, 134.4, 129.0, 118.3, 109.2, 75.7, 75.2, 73.0, 71.1, 52.1, 27.5, 26.0, 24.3; HRMS m/z calcd for C₁₄H₂₁O₅ ([M+H]⁺) 269.1407, found 269.1407 (See, e.g., Mohanrao, R.; Asokan, A.; Sureshan, K. M. Chem. Commun. 2014, 50 (51), 6707-6710.).

### Example 1.26—((3aR,7S,7aR)-7-(Allyloxy)-2,2-dimethyl-3a,4,7,7a-tetrahydrobenzo[d][1,3]dioxol-5-yl) methanol (39)

A solution of methyl 38 (0.040 g, 0.149 mmol, 1 eq) in THF (0.2 mL) was treated at −78° C. with DIBAL-H (0.298 mL, 0.298 mmol, 2 eq, 1 M in hexanes), warmed to room temperature over several hours, and stirred overnight. The reaction mixture was quenched with saturated ammonium chloride (5 mL), and extracted with CH₂Cl₂ (3×10 mL). The combined organic layers were dried (MgSO₄) and concentrated in vacuo to give 39 (0.015 g, 0.062 mmol, 42%) that was used in subsequent steps without further purification.

### Example 1.27—(1R,2R,3S)-3-(Allyloxy)-5-(hydroxymethyl)cyclohex-4-ene-1,2-diol (40)

A solution of 39 (0.038 g, 0.158 mmol) in TFA and water (80:20, 0.53 mL) was allowed to stir at room temperature for 30 min and concentrated in vacuo. to give 40 (0.011 g, 0.055 mmol, 88%) that was used in subsequent steps without further purification.

### Example 1.28—(1R,2R,3S)-3-(Allyloxy)-5-(((3,3-dimethylbutanoyl)oxy)methyl)cyclohex-4-ene-1,2-diyl bis(3,3-dimethylbutanoate) (41)

A solution of 40 (0.080 g, 0.400 mmol, 1 eq) in pyridine (1.33 mL) was treated with tert-butyl acetyl chloride (0.566, 4.00 mmol, 10 eq) and catalytic DMAP and stirred at room temperature overnight. The reaction mixture was quenched with the addition of EtOAc (5 mL), extracted with 1 M HCl (2×5 mL) and brine (5 mL), dried (MgSO₄) and concentrated under reduced pressure. The resulting residue was purified by chromatography on SiO₂ (8:1, Hexanes:EtOAc) to give 41 (0.087 g, 0.176 mmol, 71%) as a colorless oil: ¹H NMR (CDCl₃, 300 MHz) δ 5.82-5.79 (m, 1H), 5.66-5.61 (m, 2H), 5.29-5.02 (m, 3H), 4.49 (bs, 2H), 4.19-4.11 (m, 2H), 4.00-3.92 (m, 1H), 2.38-2.31 (m, 2H), 2.24 (s, 2H), 2.22 (s, 2H), 2.17 (s, 2H), 1.03 (s 9H), 1.02 (s, 9H), 1.00 (s, 9H).

### Example 1.29—(1R,2S,3S)-5-(((3,3-Dimethylbutanoyl)oxy)methyl)-3-propoxycyclohexane-1,2-diyl bis(3,3-dimethylbutanoate) (42)

A solution of 41 (0.020 g, 0.04 mmol, 1 eq) in THF (0.2 mL) was treated with Pd/C (10%, 0.004 g, 0.004 mmol) and H₂ gas and stirred at room temperature overnight. The mixture was filtered through Celite® and the solvent was removed under reduced pressure to give 42 as a 2:1 mixture of diastereomers.

The present Example further provides a suggested synthesis of (1S,2S,3S,4R,6S)-4-(acetoxymethyl)-6-propoxycyclohexane-1,2,3-triyl triacetate (43), (1S,2S,4S,6S)-2-acetoxy-4-(acetoxymethyl)-6-propoxycyclohexyl pivalate (44), and (1R,2R,4S,6S)-2-hydroxy-6-propoxycyclohexane-1,4-diyl bis(2,2-dimethylpropanoate) (45) as shown by Scheme VIII.

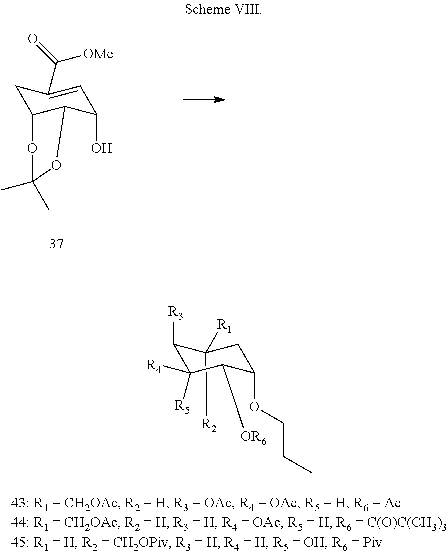

### Example 1.30—(1S,2S,3S,4R,6S)-4-(acetoxymethyl)-6-propoxycyclohexane-1,2,3-triyl triacetate (43)

Starting with 37, an analogous synthesis to Scheme VII is used to give 43 (See, e.g., Mohanrao, R.; Asokan, A.; Sureshan, K. M. Chem. Commun. 2014, 50 (51), 6707-6710.).

### Example 1.31—(1R,2R,4S,6S)-2-hydroxy-6-propoxycyclohexane-1,4-diyl bis(2,2-dimethylpropanoate) (44)

Starting with 37, an analogous synthesis to Scheme VII is used to give 44 (See, e.g., Mohanrao, R.; Asokan, A.; Sureshan, K. M. Chem. Commun. 2014, 50 (51), 6707-6710.).

### Example 1.32—(1S,2S,4S,6S)-2-acetoxy-4-(acetoxymethyl)-6-propoxycyclohexyl pivalate (45)

Starting with 37, an analogous synthesis to Scheme VII is used to give 45 (See, e.g., Mohanrao, R.; Asokan, A.; Sureshan, K. M. Chem. Commun. 2014, 50 (51), 6707-6710.).

The present Example further provides a suggested synthesis of ((1S,3S,4S,5S)-3-acetoxy-5-isobutoxy-4-pivalamidocyclohexyl)methyl acetate (47), ((1R,3R,4S,5S)-3-acetoxy-5-phenethoxy-4-pivalamidocyclohexyl)methyl acetate (48), and (1R,2S,3S,5R)-2-acetamido-3-isopropoxy-5-((pivaloyloxy)methyl)cyclohexyl azetidine-1-carboxylate (49) as shown by Scheme VIII.

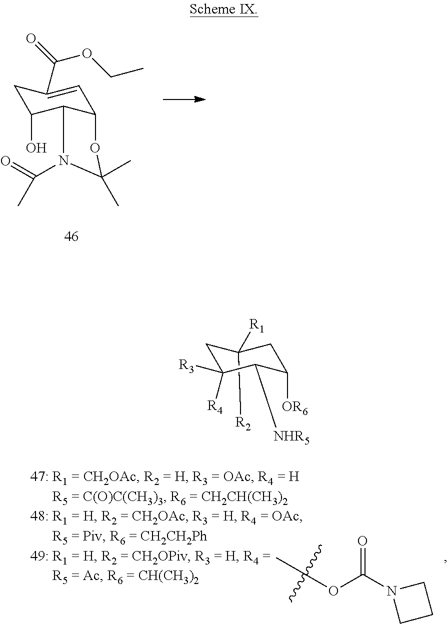

### Example 1.33—((1S,3S,4S,5S)-3-acetoxy-5-isobutoxy-4-pivalamidocyclohexyl)methyl acetate (47)

Starting with 46, an analogous synthesis to Scheme VII is used to give 47 (See, e.g., Shie, J.-J.; Fang, J.-M.; Wang, S.-Y.; Tsai, K.-C.; Cheng, Y.-S. E.; Yang, A.-S.; Hsiao, S.-C.; Su, C.-Y.; Wong, C.-H. J. Am. Chem. Soc. 2007, 129 (39), 11892-11893.).

### Example 1.34—((1R,3R,4S,5S)-3-acetoxy-5-phenethoxy-4-pivalamidocyclohexyl)methyl acetate (48)

Starting with 46, an analogous synthesis to Scheme VII is used to give 48 (See, e.g., Shie, J.-J.; Fang, J.-M.; Wang, S.-Y.; Tsai, K.-C.; Cheng, Y.-S. E.; Yang, A.-S.; Hsiao, S.-C.; Su, C.-Y.; Wong, C.-H. J. Am. Chem. Soc. 2007, 129 (39), 11892-11893.).

### Example 1.36—(1R,2S,3S,5R)-2-acetamido-3-isopropoxy-5-((pivaloyloxy)methyl)cyclohexyl azetidine-1-carboxylate (49)

Starting with 46, an analogous synthesis to Scheme VII is used to give 49 (See, e.g., Shie, J.-J.; Fang, J.-M.; Wang, S.-Y.; Tsai, K.-C.; Cheng, Y.-S. E.; Yang, A.-S.; Hsiao, S.-C.; Su, C.-Y.; Wong, C.-H. J. Am. Chem. Soc. 2007, 129 (39), 11892-11893.).

### Example 2: Compounds for Controlling Inflammatory and Immune Responses Associated with Infection

The present Example provides for approaches for controlling inflammation response according to certain embodiments of the present disclosure.

### Experimental Methods and Materials

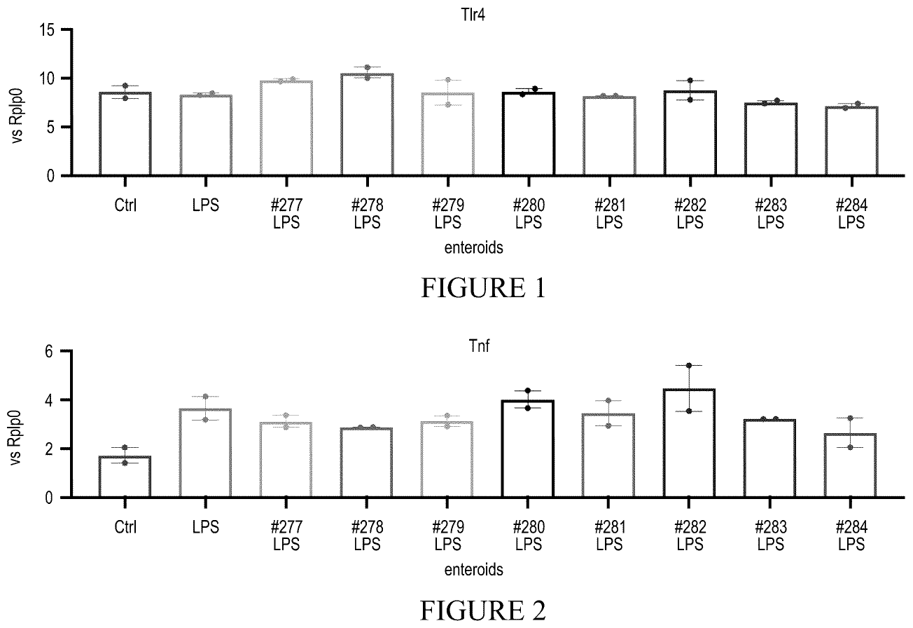

Mouse enteroids. Primary intestinal crypt cultures (enteroids) were generated from the ileum of neonatal (p7-p11) mice and maintained in Matrigel (Corning). The enteroids were digested and passed using TrypLE Express (Gibco) weekly, and used between passage 3 and 10 for all experiments. The enteroids were pre-treated with C34 analogues (20 μM, overnight) and then treated with LPS (50 μg per mL) for 4 h for further analysis. The relative mRNA expression levels of Toll-like receptor 4 (Tlr4) (FIG. 1) and tumor necrosis factor (Tnf) (FIG. 2) were shown.

Mice. C57BL/6J mice were purchased from the Jackson Laboratory, and all mice were housed in a specific pathogen free environment (ambient temperature between 2° and 25° C., humidity between 30 to 70%) on a 12-hour-light/12-hour-dark cycle with free access to water and standard rodent chow (Teklad global 18% protein rodent diets, Envigo).

Endotoxemia model. Endotoxemia was induced in neonatal pups (p11-p14) of either gender, which were randomly divided into control and test groups, by administering 5 mg per kg lipopolysaccharide (LPS) via intraperitoneal injection, and ileal samples were harvested 6 h after LPS treatments. C34 and C34 analogues were given through oral gavage 1 day prior to the LPS injection at the dose of 20 mg per kg body weight.

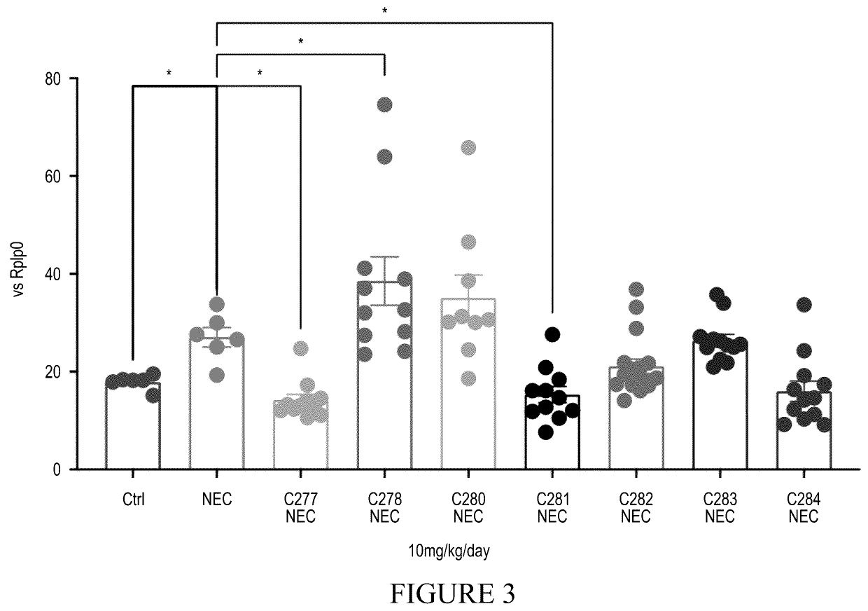

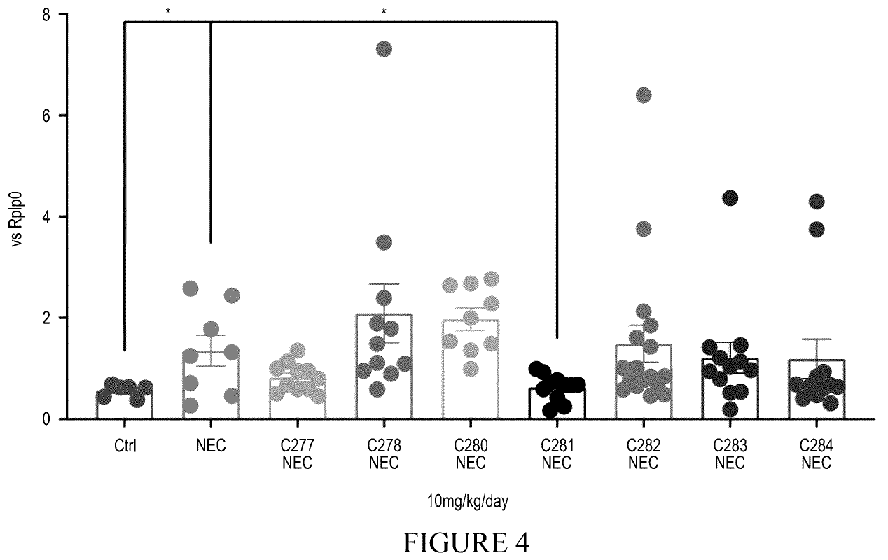

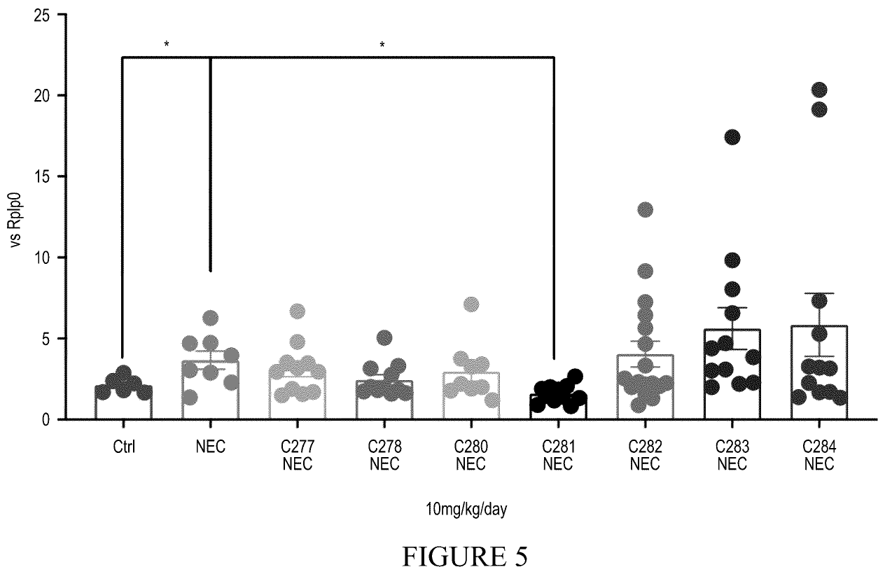

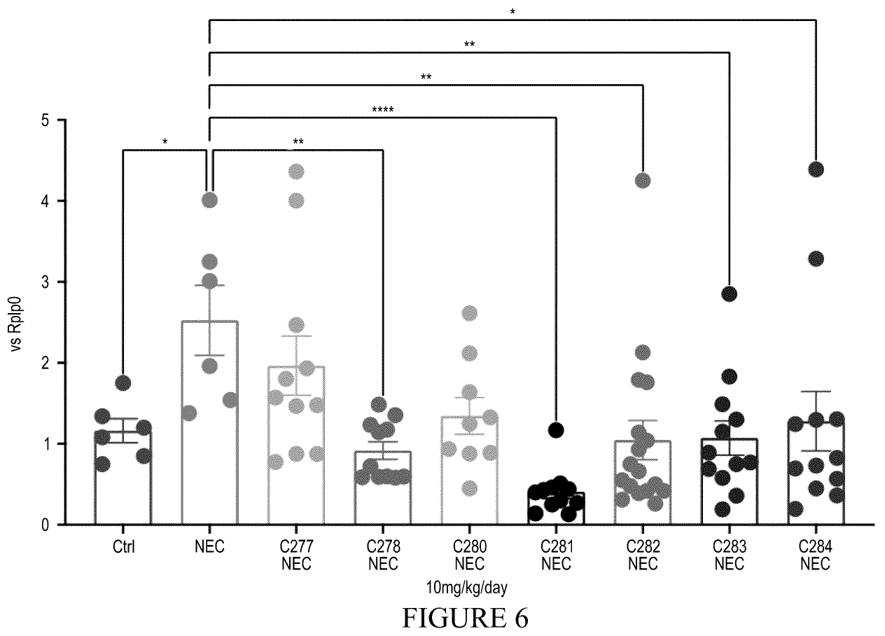

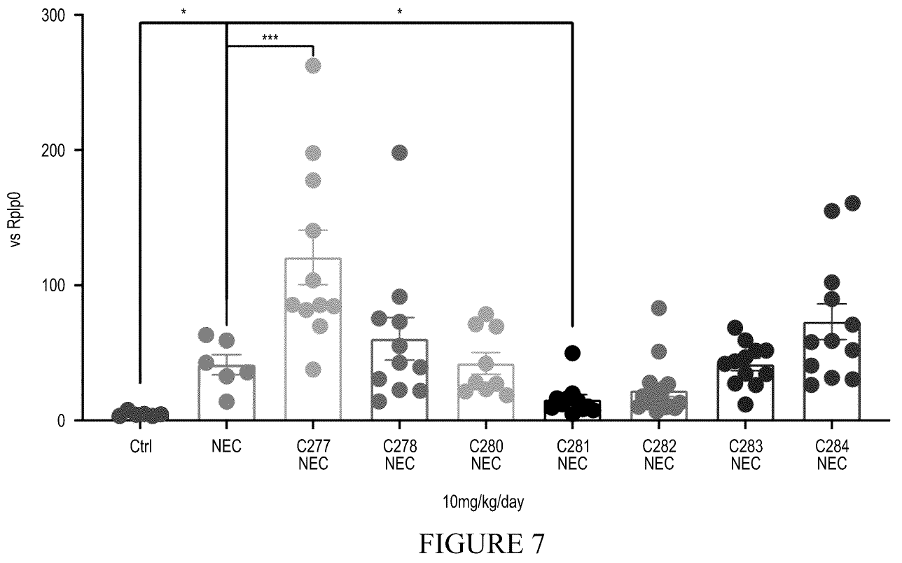

Mouse NEC model. Experimental NEC was induced in a well validated and reproducible model in 7-day-old mice of either gender, which were randomly divided into control and test groups, by gavage feeding newborn mice with formula containing Similac Advance infant formula (Abbott Nutrition): Esbilac (PetAg) canine milk replacer, 2:1 ratio, which was supplemented with enteric bacteria made from a stock created from a specimen obtained from an infant with surgical NEC five times per day. Additionally, the mice were subjected to hypoxia (5% O2-95% N2) for 10 min in a hypoxia chamber (Billups-Rothenberg) twice daily for 4 days. The C34 analogues were administered by oral gavage during the induction of NEC at the dose of 10 mg per kg body weight per day. Age-matched breast milk-fed mouse pups were used as non-NEC controls. The relative mRNA expression levels of Toll-like receptor 4 (Tlr4) (FIG. 3), interleukin 6 (Il6) (FIG. 4), interleukin 1 beta (Il1b) (FIG. 5), tumor necrosis factor (Tnf) (FIG. 6) and lipocalin 2 (Lcn2) (FIG. 7) were shown.

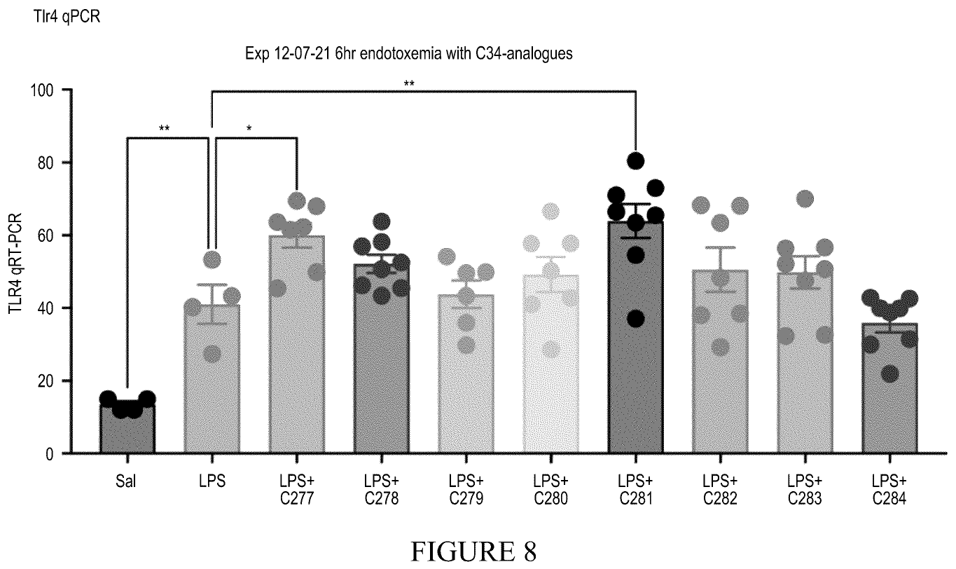

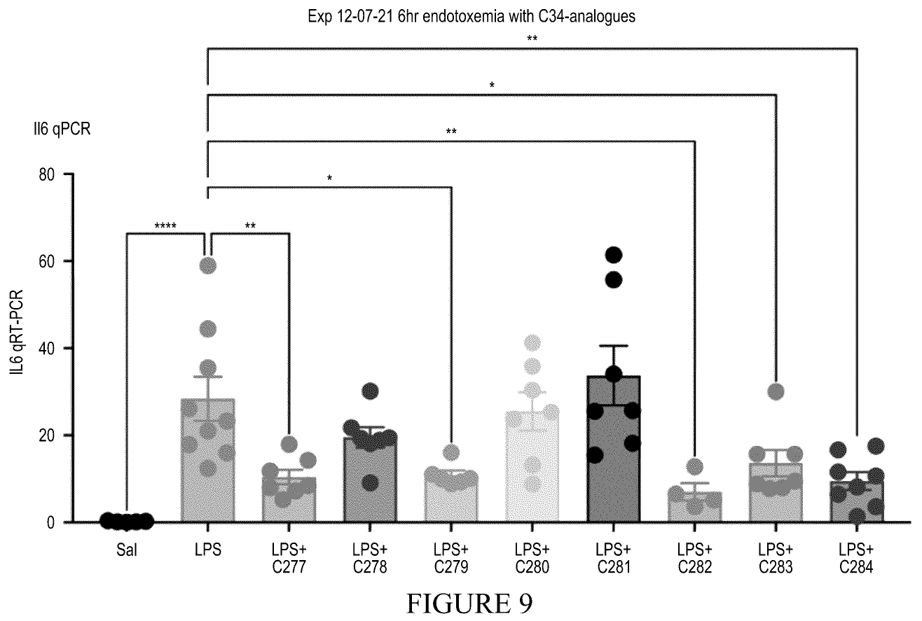

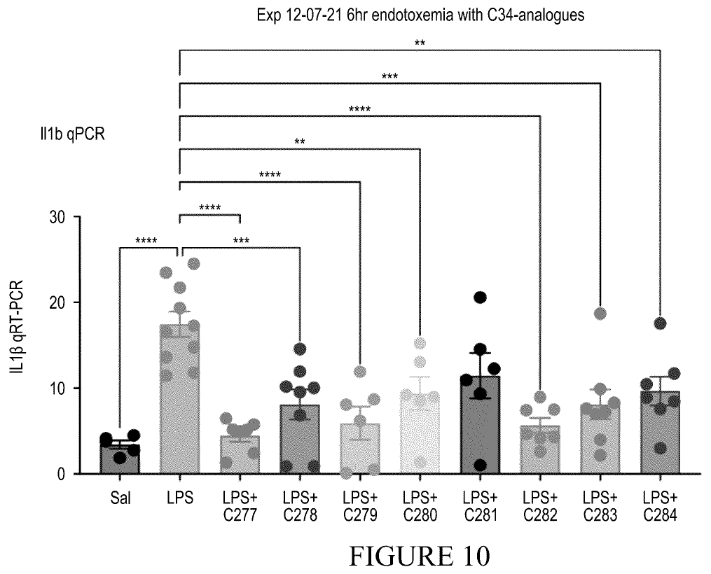

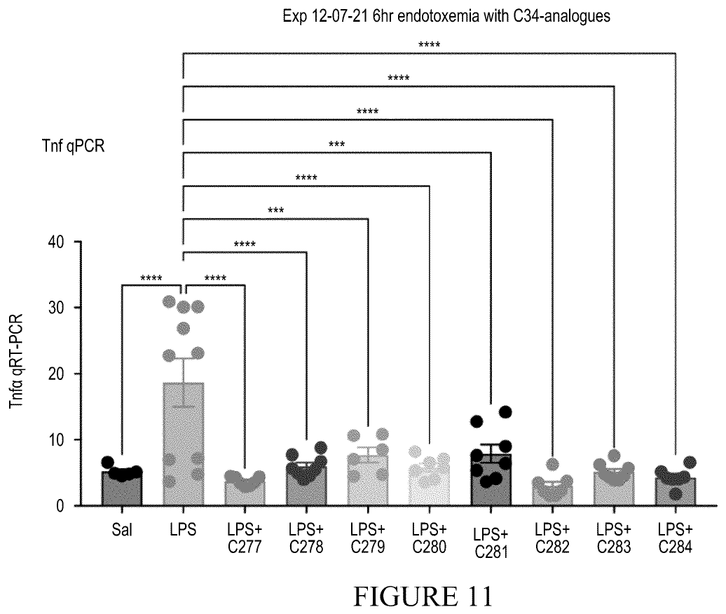

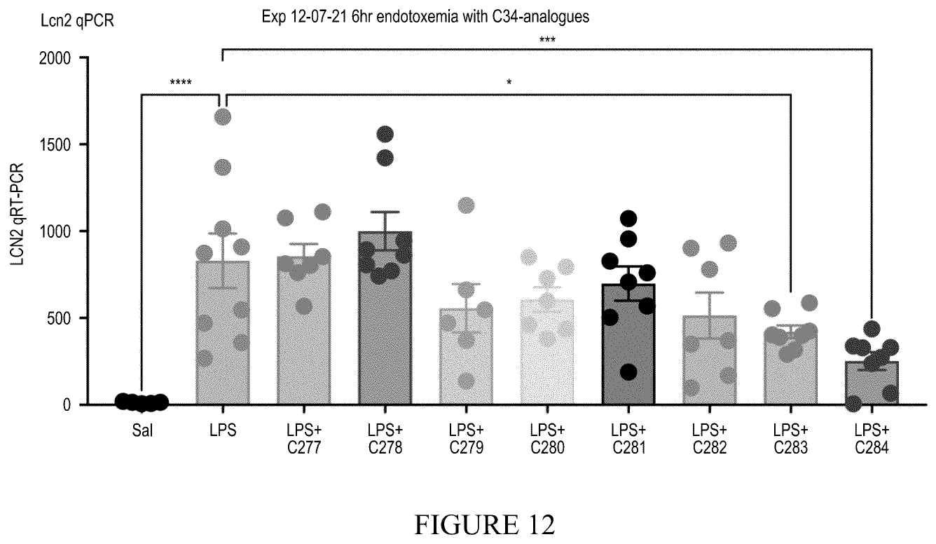

RNA isolation, cDNA synthesis and qPCR. Total RNA was isolated using the RNeasy mini kit (Qiagen) and complementary DNA was synthesized from 0.5 μg RNA using QuantiTect Reverse Transcription kit (Qiagen) following the manufacturer's protocols. The mRNA quantification was performed on the Bio-Rad CFX96 Real-Time System (Bio-Rad) using iTaq™ universal SYBR® Green supermix (Bio-Rad) and Bio-Rad CFX Manager 3.1 software was used to collect data from qRT-PCR. The relative mRNA expression levels of Toll-like receptor 4 (Tlr4) (FIG. 8), interleukin 6 (Il6) (FIG. 9), interleukin 1 beta (Il1b) (FIG. 10), tumor necrosis factor (Tnf) (FIG. 11), and lipocalin 2 (Lcn2) (FIG. 12) were normalized against the expression of the housekeeping gene ribosomal protein lateral stalk subunit P0 (Rplp0).

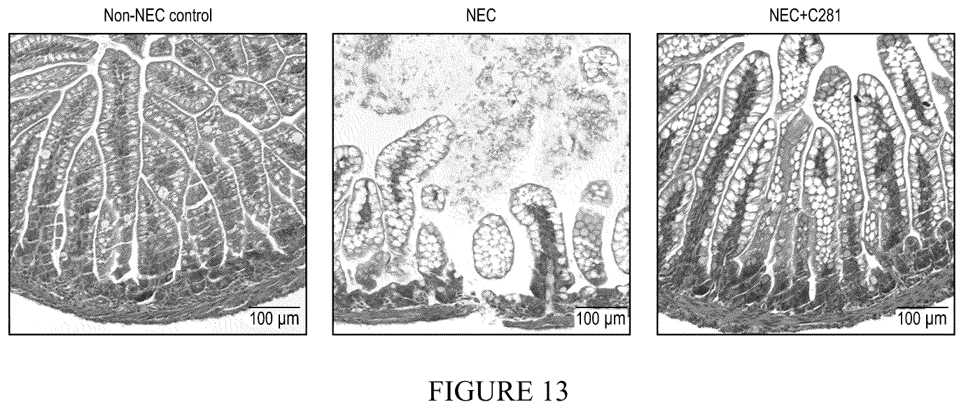

Hematoxylin and eosin (H&E) staining. The 5-μm paraformaldehyde-fixed paraffin-embedded tissue sections were rehydrated, stained in hematoxylin solution (Sigma-Aldrich), differentiated in Epredia™ Richard-Allan Scientific™ differentiating solution (Fisher Scientific), treated with Epredia™ Signature Series™ bluing reagent (Fisher Scientific), stained with eosin Y solution (Sigma-Aldrich), dehydrated, and then mounted using Permount mounting medium (Fisher Chemical) prior to imaging using a Leica DMi 8 microscope. The H&E-stained representative images of non-NEC control, NEC, and NEC+C281 groups were shown (FIG. 13).

Statistical Analysis. Transcript levels for inflammatory biomarkers were assessed by performing a one-way ANOVA statistical analysis followed by multiple comparison. Statistical significance is indicated by p-values, where *p<0.05, ***p<0.001 ****p<0.0001. Data is represented as relative mRNA expression normalized to the housekeeping gene Rplp0. Each data point represents and individual mouse.

### Results

The present examples demonstrated the effectiveness of C34 analogs in controlling inflammation and immune response associated with infection. In vitro studies were conducted using primary intestinal crypt cultures, specifically enteroids. The pre-treatment of enteroids with C34 analogs demonstrated the ability to control inflammation induced by lipopolysaccharide (LPS), as indicated by the relative mRNA expression levels of Toll-like receptor 4 (Tlr4) (FIG. 1) and tumor necrosis factor (Tnf) (FIG. 2).

The present example further evaluated the efficacy of C34 analogs in controlling inflammation in an endotoxemia mouse model. Neonatal pups were subjected to endotoxemia by administering lipopolysaccharide (LPS) via intraperitoneal injection. The effects of C34 analogs were evaluated by pre-administering them through oral gavage one day prior to the LPS injection. Ileal samples were harvested six hours after LPS treatment to assess the inflammatory response markers Toll-like receptor 4 (Tlr4) (FIG. 8), interleukin 6 (Il6) (FIG. 9), interleukin 1 beta (Il1b) (FIG. 10), tumor necrosis factor (Tnf) (FIG. 11) and lipocalin 2 (Lcn2) (FIG. 12).

Similarly, C34 analogs were evaluated in a mouse model for necrotizing enterocolitis inflammation markers Toll-like receptor 4 (Tlr4) (FIG. 3), interleukin 6 (Il6) (FIG. 4), interleukin 1 beta (Il1b) (FIG. 5), tumor necrosis factor (Tnf) (FIG. 6) and lipocalin 2 (Lcn2) (FIG. 7) were evaluated following induction of NEC, via qRT-PCR analysis. Notably, the C281 analog demonstrated a robust response. Furthermore, treatment with the C281 analog demonstrated the ability to preserve the tissue architecture of the intestinal lining in NEC mice.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, machine, manufacture, and composition of matter, methods and processes described in the specification. As one of ordinary skill in the art will readily appreciate from the disclosed subject matter of the presently disclosed subject matter, processes, machines, manufacture, compositions of matter, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

What is claimed is:

1. A compound of Formula I or stereoisomers thereof: 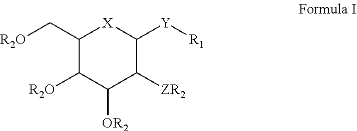
wherein X is O, Z is O or NH, Y is CH₂ or CH(R₁), R₁ is alkyl, branched alkyl, cycloalkyl, aryl, or substituted aryl, and R₂ is H or COR₃, where R₃ is selected from R₁, NHR₁, or N(R₁)₂, or
wherein X is CH₂, Z is O or NH, and Y is O, R₁ is alkyl, branched alkyl, cycloalkyl, aryl, or substituted aryl, and R₂ is H or COR₃, where R₃ is selected from R₁, NHR₁, N(R₁)₂.

2. A pharmaceutical composition comprising a compound of claim 1.

3. The pharmaceutical composition of claim 2, wherein the pharmaceutical composition is selected from the group consisting of a coated particle, a micelle, a liposome, a tablet, a capsule, a sachet, a suppository, a liquid pharmaceutical composition, and combinations thereof.

4. The pharmaceutical composition of claim 2, wherein the pharmaceutical composition further comprises an antibiotic agent, a steroid, or a non-steroidal anti-inflammatory agent, or combinations thereof.

5. The compound of claim 1, wherein the compound is selected from the group consisting of 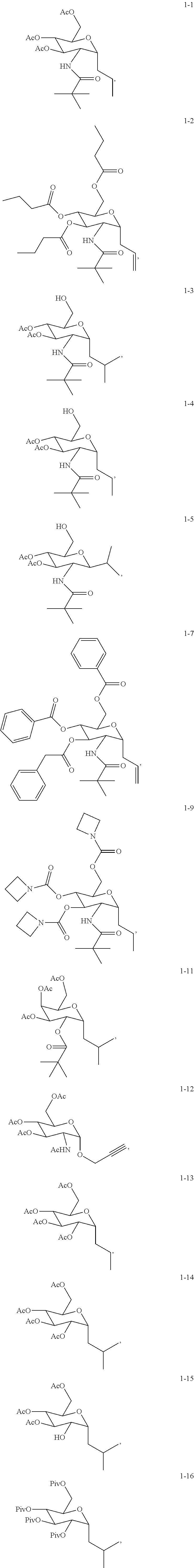 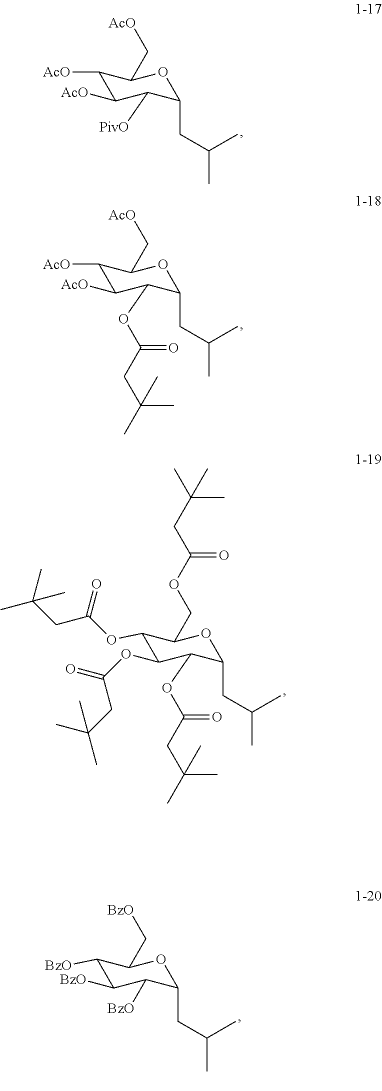
and combinations thereof.

6. A method of treating an infectious or inflammatory disorder comprising administering, to a subject in need of such treatment, an effective amount of a Toll-like receptor 4 inhibitor compound of Formula I or stereoisomers thereof: 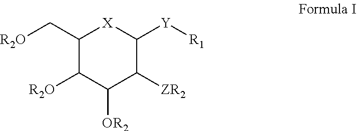
wherein X is O, Z is O or NH, Y is CH₂ or CH(R₁), R₁ is alkyl, branched alkyl, cycloalkyl, aryl, or substituted aryl, and R₂ is H or COR₃, where R₃ is selected from R₁, NHR₁, or N(R₁)₂, or
wherein X is CH₂, Z is O or NH, and Y is O, R₁ is alkyl, branched alkyl, cycloalkyl, aryl, or substituted aryl, and R₂ is H or COR₃, where R₃ is selected from R₁, NHR₁, or N(R₁)₂.

7. The method of claim 6, wherein the subject is suffering from one or more of osteoarthritis, pancreatitis, a metabolic syndrome, trauma induced systemic inflammation, acute respiratory distress syndrome, and COVID-19-induced systemic inflammation.

8. The method of claim 6, wherein the subject is a suffering from necrotizing enterocolitis.

9. The method of claim 6, wherein the Toll-like receptor 4 inhibitor is selected from the group consisting of: 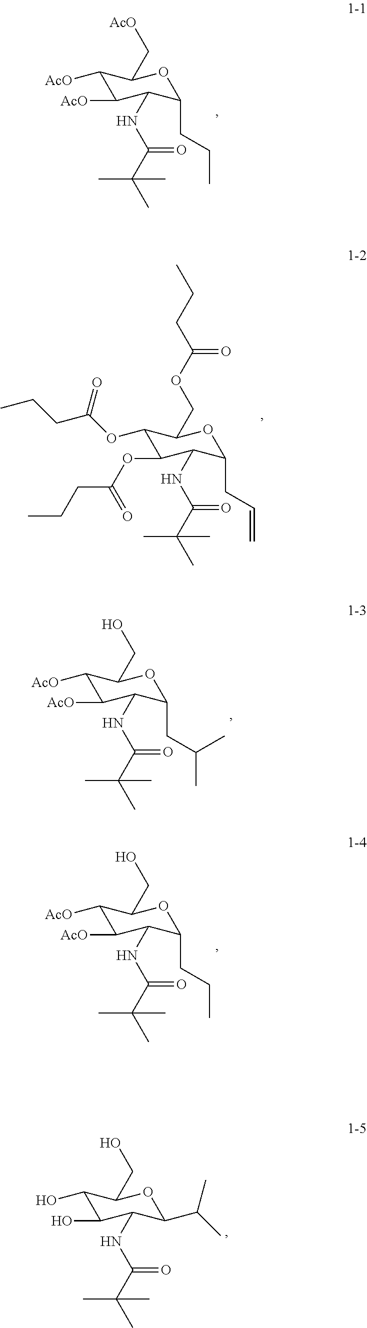 [SMILES: CCCC1OC(COC(C)=O)C(OC(C)=O)C(OC(C)=O)C1NC(=O)C(C)(C)C ; C=CCC1OC(COC(=O)CCC)C(OC(=O)CCC)C(OC(=O)CCC)C1NC(=O)C(C)(C)C ; CC(=O)OC1C(CO)OC(CC(C)C)C(NC(=O)C(C)(C)C)C1OC(C)=O ; CCCC1OC(CO)C(OC(C)=O)C(OC(C)=O)C1NC(=O)C(C)(C)C ; CC(C)C1OC(CO)C(O)C(O)C1NC(=O)C(C)(C)C] 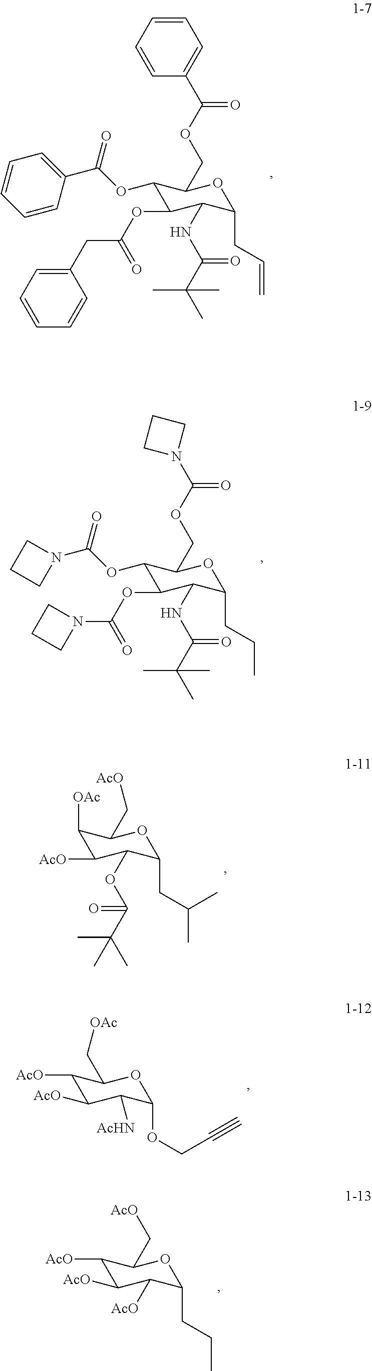 [SMILES: C=CCC1OC(COC(=O)c2ccccc2)C(OC(=O)c2ccccc2)C(OC(=O)Cc2ccccc2)C1NC(=O)C(C)(C)C ; CCCC1OC(COC(=O)N2CCC2)C(OC(=O)N2CCC2)C(OC(=O)N2CCC2)C1NC(=O)C(C)(C)C ; CC(=O)OCC1OC(CC(C)C)C(OC(=O)C(C)(C)C)C(OC(C)=O)C1OC(C)=O ; C#CCOC1OC(COC(C)=O)C(OC(C)=O)C(OC(C)=O)C1NC(C)=O ; CCCC1OC(COC(C)=O)C(OC(C)=O)C(OC(C)=O)C1OC(C)=O] 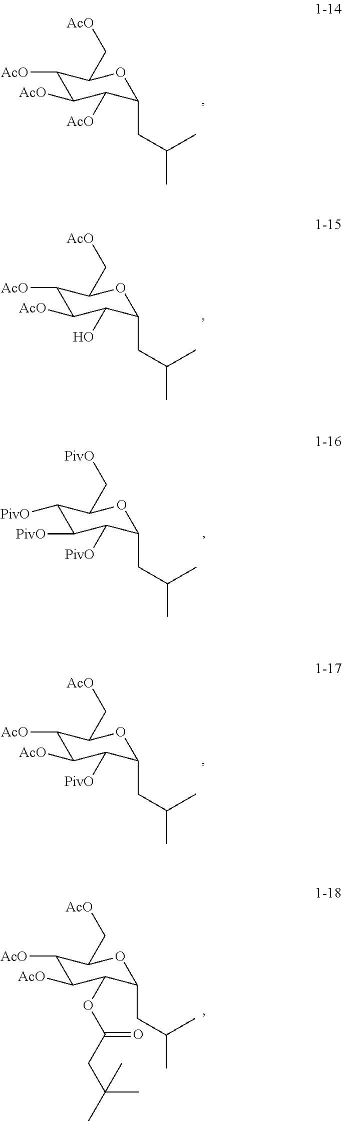 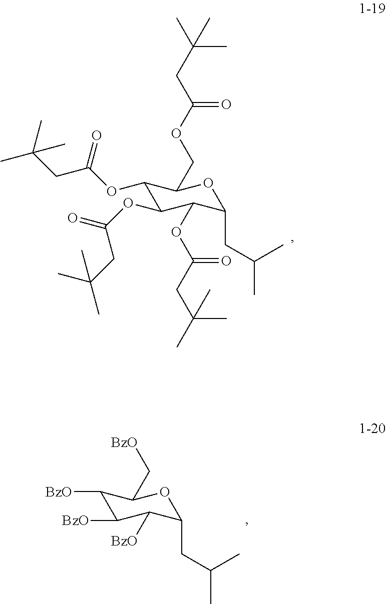
and combinations thereof.

10. A method of treating a traumatic injury in a subject comprising administering, to a subject in need of such treatment, an effective amount of a Toll-like receptor 4 inhibitor compound of claim 6 that reduces Toll-like receptor 4-induced post-traumatic injury.

11. The method of claim 10, where the traumatic injury is to an organ selected from the group consisting of the heart, the liver, the lung, the kidney, the intestine, the brain, the eye and the pancreas.

12. The method of claim 6, wherein the compound is administered to treat organ rejection after transplantation.

13. The method of claim 6 comprising administering a compound of Formula I to prevent necrotizing enterocolitis in a premature infant.
